SEQUENCE LISTING (30 sequences)

SEQ ID NO: 1 - DNA, 20 bp, Artificial Sequence
  Synthesized
ggaaacaggtttgtcatggc

SEQ ID NO: 2 - DNA, 20 bp, Artificial Sequence
  Synthesized
gcgagaaaccagaaggagac

SEQ ID NO: 3 - DNA, 22 bp, Artificial Sequence
  Synthesized
ggtgaaggtcggagtcaacgga

SEQ ID NO: 4 - DNA, 22 bp, Artificial Sequence
  Synthesized
gagggatctcgctcctggaaga

SEQ ID NO: 5 - DNA, 22 bp, Artificial Sequence
  Synthesized
ttgggtcttgttcactccacgg

SEQ ID NO: 6 - DNA, 24 bp, Artificial Sequence
  Synthesized
cctctttcaggtcactttggtagg

SEQ ID NO: 7 - DNA, 21 bp, Artificial Sequence
  Synthesized
agctatttgaggaactcggtg

SEQ ID NO: 8 - DNA, 20 bp, Artificial Sequence
  Synthesized
catcttttgtgccgtggttg

SEQ ID NO: 9 - DNA, 20 bp, Artificial Sequence
  Synthesized
ggtcaaggctacggaaagtg

SEQ ID NO: 10 - DNA, 20 bp, Artificial Sequence
  Synthesized
tcttcagcaaagtcagaggc

SEQ ID NO: 11 - DNA, 20 bp, Artificial Sequence
  Synthesized
gagggagtcaaatagcggtg

SEQ ID NO: 12 - DNA, 20 bp, Artificial Sequence
  Synthesized
atctgaacgaaggctggaag

SEQ ID NO: 13 - DNA, 23 bp, Artificial Sequence
  Synthesized
ctgaagacaatggcaagtaatgg

SEQ ID NO: 14 - DNA, 22 bp, Artificial Sequence
  Synthesized
actgcattaagacgtggatcag

SEQ ID NO: 15 - DNA, 20 bp, Artificial Sequence
  Synthesized
gagtgcaaaaggaaagccag

SEQ ID NO: 16 - DNA, 21 bp, Artificial Sequence
  Synthesized
atgataccagcaatagcgtcc

SEQ ID NO: 17 - DNA, 21 bp, Artificial Sequence
  Synthesized
gtgacccttgagcctttagag

SEQ ID NO: 18 - DNA, 21 bp, Artificial Sequence
  Synthesized
acaccatcaatctcaacctcg

SEQ ID NO: 19 - DNA, 20 bp, Artificial Sequence
  Synthesized
gagataccgacgcaacgact

SEQ ID NO: 20 - DNA, 20 bp, Artificial Sequence
  Synthesized
ttggtttgctgctcgtttgg

SEQ ID NO: 21 - DNA, 20 bp, Artificial Sequence
  Synthesized
attgcactgcagtttgccag

SEQ ID NO: 22 - DNA, 20 bp, Artificial Sequence
  Synthesized
ggtatgactcgtaagccggg

SEQ ID NO: 23 - DNA, 19 bp, Artificial Sequence
  Synthesized
cgggtaccgcgtcaatttc

SEQ ID NO: 24 - DNA, 19 bp, Artificial Sequence
  Synthesized
tggcggcgaaccctataaa

SEQ ID NO: 25 - DNA, 24 bp, Artificial Sequence
  Synthesized
acgtcagccggaagtgaaagaaga

SEQ ID NO: 26 - DNA, 24 bp, Artificial Sequence
  Synthesized
agcggacagtcaattccagatcgt

SEQ ID NO: 27 - DNA, 22 bp, Artificial Sequence
  Synthesized
tgacaacgacggtggataatag

SEQ ID NO: 28 - DNA, 21 bp, Artificial Sequence
  Synthesized
gccaatgggtattgctgaaac

SEQ ID NO: 29 - DNA, 21 bp, Artificial Sequence
  Synthesized
atcagatatctcgcaagctcg

SEQ ID NO: 30 - DNA, 21 bp, Artificial Sequence
  Synthesized
caactccatctcccattcctg

(19) United States
(12) Patent Application Publication
(10) Pub. No.: US 2021/0330603 A1
(43) Pub. Date: Oct. 28, 2021

(54) METHODS OF USING CANNABINOIDS FOR INHIBITING INDUCTION OF VIRULENCE IN ENTERIC PATHOGENS

(71) Applicants: THE REED INSTITUTE, Portland, OR (US); THE BOARD OF REGENTS OF THE UNIVERSITY OF TEXAS SYSTEM, Austin, TX (US)

(72) Inventors: Jay Mellies, Portland, OR (US); Vanessa Sperandio, Austin, TX (US)

(21) Appl. No.: 17/184,350

(22) Filed: Feb. 24, 2021

Related U.S. Application Data
(60) Provisional application No. 62/980,728, filed on Feb. 24, 2020.

(51) Int. Cl. A61K 31/05; A61K 31/232; A61K 45/06; A61P 31/04

(52) U.S. Cl. CPC A61K 31/05; A61P 31/04; A61K 45/06; A61K 31/232

(57) ABSTRACT

Methods of treating a subject having a gram negative bacterial infection by administering a cannabinoid such as 2-AG,CBD, the MgI inhibitor JZL184, or arachidonic acid to the subject are provided. Methods of prophylactically treating a subject at risk of developing a gram negative bacterial infection and for reducing the bacterial virulence in an infected subject are also provided.

### CROSS-REFERENCE TO RELATED APPLICATIONS

This application claims the benefit of U.S. provisional patent application No. 62/980,728 filed Feb. 24, 2020 and entitled “METHODS OF USING CANNABINOIDS FOR INHIBITING INDUCTION OF VIRULENCE IN ENTERIC PATHOGENS,” the entire contents of which are hereby incorporated herein by reference in its entirety.

### STATEMENT REGARDING FEDERALLY SPONSORED RESEARCH

This invention was made with government support under Grant No. RO-37 A1053067 awarded by the United States National Institutes of Health. The government has certain rights in the invention.

### TECHNICAL FIELD

This disclosure relates generally to methods of inhibiting gram negative bacterial virulence. More specifically, this disclosure relates to methods of using cannabinoids to reduce the virulence of enteric pathogens that are infecting a subject.

### BACKGROUND

The gastrointestinal (GI) tract harbors a complex community of endogenous microbes that serve as an essential component in maintaining intestinal homeostasis.¹ The metabolic and physiological activities of host and microbial cells generate an intestinal microenvironment with a diverse milieu of small molecules derived from host, microbial and external sources. Invading enteric pathogens have evolved mechanisms to sense these chemical cues and to integrate this biochemical information into regulating their signaling cascades to maximize success of initial intestinal colonization, rapid niche formation and subsequent transmission to the next host.²

The endocannabinoid system is composed of lipid-based signaling molecules known as endocannabinoids, which modulate GI physiology and immunity.^{3,4} Lipases encoded within bacterial genomes are capable of degrading endocannabinoids such as 2-arachidonoyl-glycerol (2-AG),^{5,6} which suggest that bacteria may respond to this class of lipid neurotransmitters. 2-AG signaling in the host attenuates pro-inflammatory immune responses in chemical models of colitis.⁷ However, the effects of endocannabinoids such as 2-AG in the context of infectious colitis have not been explored.

### BRIEF DESCRIPTION OF THE FIGURES

FIG. 1. Mice with elevated 2-AG levels are resistant to infectious colitis. a, Schematics of 2-arachidonoyl (2-AG) structure, 2-AG hydrolysis in mammalian tissues and 2-AG gradients in the intestines. SI, small intestines. Col, colon. MgII, monoacylglycerol lipase. b-c, Quantification of 2-AG in colon tissues harvested from b, PBS-treated (n=6-8) and c, C. rodentium (CR) infected (n=10-13) MgII^{+/+}, MgII^{+/-} or MgII^{-/-} mice. d-f, CR burdens in MgII^{+/+} or MgII^{-/-} mice in d, feces (n=14-18), e, cecal tissues (n=14-18) and f, distal colon (DC) tissues (n=8-10). g, Gross pathology scores of ceca and colons harvested from CR infected MgII^{+/+} or MgII^{-/-} mice (n=8-10). h-i, Cecal transcript levels of h, Nos2 and i, Mip2a in PBS treated (n=3) and CR infected (n=14-18) MgII^{+/+} or MgII^{-/-} mice. j-k, Histopathology scores of j, ceca and k, colons from PBS treated (n=3) and CR infected (n=8-10) MgII^{+/+} or MgII^{-/-} mice. l, Representative H&E histology of ceca from PBS treated and CR infected MgII^{+/+} or CR infected MgII^{-/-} mice. Scale bar, 100 μm. m-n, Transcript levels of LEE-encoded n, espA and o, tir in cecal contents from CR infected MgII^{+/+} or MgII^{-/-} mice (n=9-13). o, Schematic depicting the genetic organization of the LEE pathogenicity island in A/E pathogens. b-f, h-i, m-n, Lines are at the mean. P-values were determined by Student's unpaired t-test or one-way ANOVA with the Bonferroni's multiple comparison test. g, j-k, Lines are at the median. P-values were determined by Mann-Whitney or Kruskal-Wallis with the Dunn's multiple comparison test. *p<0.05, **p<0.01, ***p<0.001, ****p<0.0001.

FIG. 2. 2-AG inhibits LEE-encoded T3SS activity in A/E pathogens. EHEC or C. rodentium (CR) were grown microaerobically under LEE-inducing conditions (DMEM-low glucose) in the presence of 2-AG or the vehicle control (V). a, Differentially expressed KEGG pathways in vehicle-versus 2-AG-treated EHEC at mid-log phase as assessed by RNA-seq analysis. n=4 per group. b, Schematic of the LEE-encoded T3SS needle apparatus. c, CR secretion of the translocon components EspA and EspB at late-log phase as assessed by Western blots. Blots are representative of more than 3 independent experiments. d, Protein expression of EspA, EspB and the effector Tir in lysates from EHEC at late-log phase as assessed by Western blots. Blots are representative of 3 independent experiments. e-f, EHEC secretion of EspA and EspB at late-log phase in response to e, 2-AG and f, 2-AG or 1-AG as assessed by Western blots. Blots are representative of at least 3 independent experiments. LC, loading control. g, EHEC was treated with 10 μM 2-AG or the vehicle control prior to infection of HeLa cells. Representative confocal microscopy images of epithelial pedestal formation (white arrowheads) by m-Cherry expressing EHEC. DNA (blue) is stained with DAPI and actin (green) is stained with FITC-phalloidin. Images at 40×. h, Percentage of epithelial cells with EHEC pedestals. i, Quantity of EHEC pedestals per infected cell. At least 275 cells in 17 fields at 40× were enumerated for each group. Data are represented as the mean±SEM from three independent experiments. P-values were determined by Mann-Whitney. ***p<0.001.

FIG. 3. 2-AG inhibition of pathogenic T3SS activity is conserved in S. Typhimurium. a, Schematic of the contributions of the SPI-1 and SPI-2 genomic islands to Salmonella pathogenesis. b-e, S. Typhimurium (ST) was grown aerobically under SPI-1-inducing conditions (LB broth) in the presence of 10 μM 2-AG or the vehicle control (V). b-d, Transcript levels at late-log phase of SPI-1-encoded b, hilD, c, sipA and d, sopB. e, ST was treated with 10 μM 2-AG or the vehicle control prior to infection of HeLa cells. Epithelial invasion of vehicle- or 2-AG-treated ST (WT) as assessed by quantitative culture. The SPI-1-inactived ST mutant ΔinvA (Δ) served as a negative control. f-g, ST was grown aerobically under SPI-2-inducing conditions (N9 minimal medium) in the presence of 10 μM 2-AG or the vehicle control (V). Transcript levels at 3 hours of SPI-2-encoded f, ssaV and g, sseF. h, Intramacrophagic survival of ST in the presence of 2-AG or the vehicle control (V). i, Survival of streptomycin-treated MgII^{+/+} or MgII^{-/-} mice following infection with ST (n=10-11 for ST WT, n=3-5 for ST Δ). P-values were determined by the Mantel-Cox test. b-h, Data are represented as the mean±SEM. P-values were determined by Student's unpaired t-test or one-way ANOVA with the Bonferroni's multiple comparison test. *p<0.05, **p<0.01, ***p<0.001.

FIG. 4. 2-AG mediates its anti-virulence effects by counteracting activation of the bacterial adrenergic receptor QseC. a-d, EHEC or CR WT or ΔqseC were grown microaerobically under LEE-inducing conditions (DMEM-low glucose) in the presence of 10 μM 2-AG or the vehicle control (V). a, Protein expression of EspB and Tir in lysates from EHEC at late-log phase as assessed by Western blots. b, EHEC secretion of EspB at late-log phase as assessed by Western blots. LC, loading control. c, Transcript levels in EHEC at late-log phase of the LEES-encoded tir. d, Transcript levels in CR at late-log phase of the LEES-encoded eae. e, Autophosphorylation of QseC in liposomes in the presence or absence of 2-AG. f, Autophosphorylation of QseC in liposomes in the presence or absence of 6 μM 2-AG, 50 μM epinephrine (Epi), or 2-AG and epinephrine (both) after 30 minutes. g, Autophosphorylation of QseC in liposomes in the presence or absence of 6 μM 2-AG, or 2-AG and the CBR1 inhibitor AM251 (antiCB1) after 30 minutes. h, C57131/6 mice were infected with CR WT or its qseC isogenic mutant. CR burdens in feces collected at days 4 and 7 post infection (n=4). i, CR WT (n=14-17) and qseC (n=16) burdens in MgII^{+/+} or MgII^{-/-} mice in cecal tissues. j, Gross pathology scores of ceca and colons from infected MgII^{+/+} or MgII^{-/-} mice at day 10 post infection (n=8-10 (WT); 16 (qseC)). k, EHEC WT and ΔfadL were grown microaerobically under LEE-inducing conditions (DMEM-low glucose) in the presence of 10 μM 2-AG or the vehicle control (V). EHEC secretion of EspA and EspB at late-log phase as assessed by Western blots. LC, loading control. l, Schematic of proposed model depicting 2-AG sensing in A/E pathogens. c-g, Data are represented as the mean or the mean±SEM. P-values were determined by the Student's unpaired t-test. h-j, Lines are at the median. P-values were determined by Mann-Whitney. i-j, Data from mice infected with CR WT (3 independent cohorts) are depicted in FIG. 1, with one cohort run together with CR qseC. Three independent cohorts were run for CR qseC. *p<0.05, ***p<0.001, ****p<0.0001.

FIG. 5. C. rodentium infection in MgII^{+/+} and MgII^{-/-} mice. a, CR burdens in MgII^{+/+} or MgII^{-/-} mice in feces. Lines are at the mean. P-values were determined by the Student's t-test. b, Lengths of colons from CR infected MgII^{+/+} or MgII^{-/-} mice. Lines are at the median. P-values were determined by Mann-Whitney.

FIG. 6. Colonic levels of arachidonic acid and other MAGs are unchanged in MgII^{-/-} mice. a, Relative concentrations of arachidonic acid (20:4(ω6)) in the colons of uninfected MgII^{+/+} and MgII^{-/-} mice. b, Relative concentrations of monoacylglycerols (MAGs) in the colons of uninfected MgII^{+/+} and MgII^{-/-} mice. c, Relative concentrations of arachidonic acid (20:4(ω6)) in the colons of CR infected MgII^{+/+} and MgII^{-/-} mice. d, Relative concentrations of monoacylglycerols (MAGs) in the colons of CR infected MgII^{+/+} and MgII^{-/-} mice. Lines are at the mean. P-values were determined by one-way ANOVA and the Bonferroni's multiple comparison test. **p<0.01, ****p<0.0001.

FIG. 7. The intestinal microbiotas in MgII^{+/+} and MgII^{-/-} mice are similar in composition. a, b, Fecal samples were collected for 16S rRNA profiling from 8-week old MgII^{-/-} mice and MgII^{+/+} littermate controls (n=4 mice per group). OTU differences at the a, phyla and b, order levels prior to infection with C. rodentium.

FIG. 8. 2-AG inhibits transcription of LEE-encoded genes and non-LEE encoded effectors. a-g, EHEC was grown microaerobically under LEE-inducing conditions (DMEM-low glucose) in the presence of 10 μM 2-AG or the vehicle control (V). a, Ten most downregulated genes in vehicle versus 2-AG-treated EHEC at mid-log phase as assessed by RNA-seq analysis. n=4 per group. FC, fold change. P-val adj, adjusted p-value. b-f, Targeted qPCR of LEE-encoded genes in EHEC at late log phase. g, Targeted qPCR of stx2a, which encodes Shiga toxin, in EHEC at late-log phase. Data are represented as the mean±SEM from at least three independent experiments. P-values were determined by Student's unpaired t test. h, C. rodentium was grown aerobically under LEE-inducing conditions (DMEM-low glucose) in the presence or absence of 20 μM 2-AG. Targeted qPCR of LEE-encoded genes ler and eae and the non-LEE encoded effector nleA at late-log phase. Data are represented as the mean±SEM. n=3 biological replicates. P-values were determined by 2-way ANOVA. Pairwise comparisons by Bonferroni post test. *p<0.05, **p<0.01, ***p<0.001.

FIG. 9. 2-AG inhibits secretion of LEE-encoded effectors and translocon components. CR or EHEC were grown microaerobically under LEE-inducing conditions in the presence of 10 μM 2-AG or the vehicle control (V). a-b, CR secretion of a, EspA and b, EspB as assessed by Western blots and densitometry. c-d, EHEC secretion of c, EspA and d, EspB as assessed by Western blots and densitometry. e-g, Protein expression of e, EspA, f, EspB and g, the effector Tir in lysates from EHEC as assessed by Western blots and densitometry. Data are represented as mean±SEM from at least 3 independent experiments. P-values were determined by Student's unpaired t test or one-way ANOVA with the Bonferroni post test. *p<0.05, **p<0.01, ***p<0.001, ****p<0.0001.

FIG. 10. The endocannabinoid anandamide does not modulate LEE activity in EHEC. EHEC was grown microaerobically under LEE-inducing conditions (DMEM-low glucose) in the presence of anandamide (100 or 200 nM) or the vehicle control (V). EHEC secretion of the translocon components EspA and EspB at late-log phase as assessed by Western blots. Blots are representative of 3 independent experiments. LC, loading control.

FIG. 11. 2-AG does not alter growth kinetics of A/E pathogens. C. rodentium or EHEC were grown microaerobically under LEE-inducing conditions (DMEM-low glucose) in the presence of 10 μM 2-AG or the vehicle control (Veh). a-b, Growth curves of a, EHEC and b, C. rodentium. c, Samples were collected after 4 hours of growth and C. rodentium CFUs were enumerated by quantitative bacterial culture. Data are represented as the mean±SEM of three independent experiments.

FIG. 12. 2-AG limits SipA secretion in S. Typhimurium. S. Typhimurium was grown aerobically under SPI-1-inducing conditions in the presence of 10 μM 2-AG or the vehicle control (Veh).

FIG. 13. 2-AG does not alter growth kinetics of S. Typhimurium. Growth curves of S. Typhimurium (ST) grown aerobically under SPI-1-inducing conditions in the presence of 10 μM 2-AG or the vehicle control (Veh).

FIG. 14. Fecal burdens of S. Typhimurium in MgII^{+/+} and MgII^{-/-} mice. Fecal ST burdens in MgII^{+/+} or MgII^{-/-} mice following streptomycin treatment. Lines are at the mean. P-values were determined by the Student's t-test.

FIG. 15. 2-AG inhibits adrenergic histidine kinases. a-b, Autophosphorylation of CpxA in liposomes in the presence of 8 μM arachidonic acid (AA), 10 μM 2-AG, 500 μM indole (positive control) or the vehicle control (top) after a, 120 seconds orb, 10 minutes. Commassie gel of CpxA (lower panel, loading control). c-d, EHEC ΔfusK or ΔtorS were grown aerobically under LEE-inducing conditions in the presence or absence of 20 μM 2-AG. Targeted qRT-PCR of LEE-encoded tir. Data are represented as mean±SEM. P-values were determined by Student's unpaired t test. ***p<0.001.

FIG. 16. Dose response of C. rodentium infection in C57Bl/6 WT mice. C57Bl/6 WT mice were orally inoculated with 10⁷, 10⁸ or 10⁹ CFUs of C. rodentium. Fecal burdens of C. rodentium at days 4 or 7 days post infection. Each data point represents an individual mouse. Lines are at the median.

FIG. 17. Deletion of fadL abrogates EHEC sensing of 2-AG. EHEC WT or ΔfadL were grown microaerobically under LEE-inducing conditions (DMEM-low glucose) in the presence of 10 μM 2-AG or the vehicle control (V). a-b, EHEC secretion of a, EspA and b, EspB at late-log phase as assessed by Western blots and densitometry. Data are represented as mean±SEM from at least 3 independent experiments. P-values were determined by one-way ANOVA with the Bonferroni multiple comparison test. *p<0.05, **p<0.01, ****p<0.0001.

FIG. 18. Deletion of fadL attenuates C. rodentium fitness in the colon. C57Bl/6 WT mice were infected with C. rodentium WT or the fadL isogenic mutant. a, C. rodentium burdens in feces collected at days 2, 4 or 6 days post infection. b, C. rodentium burdens in cecal tissues harvested at days 6 or 8. Each data point represents an individual mouse. Lines are at the median. P-values were determined by Mann-Whitney. *p<0.05, **p<0.01.

FIG. 19. Downregulation of EHEC espA grown in LG-DMEM (OD ˜0.7-0.8) in the presence of 50 μM CBD (Enjoy CBD Extract purchased from Khaleafa Dispensary, Portland, Oreg.). The averages were summated from technical replicates of the 86-24 wild-type EHEC strain (a) and the ΔqseC EHEC deletion strain (b), conducted in triplicate and analyzed using a one-tailed T-Test (0.00490) and (0.0161) respectively. The target gene Ct baseline was established using rpoA as an internal control for the ΔΔCt method analysis. Error Bars represent the standard error of mean for the trials.

FIG. 20. EspA expression diminished in the presence of CBD. EHEC 86-24 or the ΔqseC deletion strain were grown to OD₆₀₀=0.800 in low glucose DMEM with CBD concentrations as indicated. A 1 ml sample of bacteria was pelleted and resuspended in 10 μL 1× Laemli buffer. Proteins were separated, transferred, and blot probed with anti-EspA (1:1000) and then secondary antibody GAR-AP (1:1000). EspA band is indicated by arrow; the cross-reactive band running at ˜30 kDa served as a loading control.

FIG. 21. Colony counts from CBD post-infection challenge. All counts enumerated on plates selecting for C. rodentium using nalidixic acid. Represented as log scaled CFUs/ml normalized per gram of feces. Statistically significant differences between the two treatment groups using Mann-Whitney U analysis represented with an asterisk (one-tailed, alpha=0.05). Counts were analyzed using biological replicates and black line in each column represents median of treatment group. Asterisks indicate statistical significance alpha <0.05.

FIG. 22. Growth curves for Staphylococcus aureus (left side graphs) and EHEC (right side graphs) in the presence of AA (top graphs) and AG (bottom graphs).

FIG. 23. Pharmacological inhibition of MgI decreases colonization of mouse cecum tissues by C. rodentium.

FIG. 24. Expression of fadL in C. rodentium recovered from cecal contents. EHEC WT, ΔfadL or ΔqseC was grown microaerobically under LEE-inducing conditions (DMEM-low glucose) in the presence of 8 μM arachidonic acid (AA), 10 μM 2-AG or the vehicle control (V). a, Secreted EspA and EspB (top) and lysate-associated Tir and EspA (bottom) at late-log phase as assessed by Western blots. Blots are representative of at least 3 independent experiments. b, EspA and EspB secretion by EHEC ΔqseC at late-log phase as assessed by Western blots. Blots are representative of at least 3 independent experiments.

### DETAILED DESCRIPTION

Because of the growing problem of antibiotic resistances and the limited therapy options for certain bacterial infections, the inventors have pursued novel means for treating ongoing microbial threats to human health, including enterohemorrhagic strains of E. coli and Salmonella enterica.

As described below, a commercially available CBD product reduced expression of genes necessary for disease caused by E. coli O157:H7. A single dose of CBD administered one-day post-infection inhibited the ability of the attaching and effacing pathogen Citrobacter rodentium to colonize the intestines of mice. These results provide promising support for the use of CBD and other cannabinoids as a safe, non-toxic intervention for human infectious disease.

The present disclosure provides methods for treating a subject having a gram negative bacterial infection. In certain embodiments, such methods comprise administering a pharmaceutical composition comprising a cannabinoid and a pharmaceutically acceptable carrier to the subject. The pharmaceutical composition may be administered in a therapeutically effective amount. A “therapeutically effective” amount is an amount sufficient to reduce a pathological effect or symptom of the gram negative bacterial infection. In certain embodiments, the therapeutically effective amount of the cannabinoid is between about 1-30 mg/kg, about 1-25 mg/kg, about 1-20 mg/kg, about 1-15 mg/kg, about 1-10 mg/kg, about 1-5 mg/kg, or about 3-4 mg/kg body weight.

In some embodiments, the cannabinoid comprises cannabidiol (CBD). Marijuana is a Schedule I drug according to United States government. While a number of states have made marijuana available for medical use (33 states and Washington D.C.), with some allowing its use for recreational purposes (10 states and Washington D.C.), the ability to conduct research on these compounds for potential drug design remains limited.⁴⁴ However, CBD is one of the most widely available cannabinoids in the United States as it can be legally purchased when isolated from low- or zero-tetrahydrocannabinolic acid (THC) hemp. Marketed for a number of ailments, it has been billed as an anti-inflammatory, pain reliever, and anti-anxiety therapeutic.⁴⁴ Furthermore, the FDA has approved CBD isolated from marijuana as an active ingredient in drugs used to combat severe forms of epilepsy in children such as Lennox-Gastaut syndrome and Dravet syndrome.⁴⁵ To corroborate its efficacy and supposedly low-risk factor, the World Health Organization offered the statement saying “CBD exhibits no effects indicative of any abuse or dependence potential . . . . To date, there is no evidence of recreational use of CBD or any public health related problems associated with the use of pure CBD.”⁴⁶

Without being bound by any particular theory, CBD is thought to indirectly act at the CB1 receptors and to have a low affinity for CB2 receptors, both of which are activated by THC and have a role in the psychoactive effects. CBD has a high lipophilicity and tends to accumulate in adipose tissue.⁴⁶ Some research has shown that CBD produces an antagonistic effect upon a CB2 agonist (CP55940). This effect is thought to have allosteric effects as a non-competitive inhibitor at the CB2 receptor.⁴⁷ It has also been shown that CBD may act as a negative allosteric modulator of the CB1 receptor, thus antagonizing the effects of THC.⁴⁸

In some embodiments, the gram negative bacterial infection is a GI tract infection. Because of the proven safety record and lack of psychoactive effects compared to THC, CBD may have potential to mitigate the pathogenesis of EHEC and other, type III secretion system encoding Gram-negative pathogens, such as Salmonella enterica, Francisella tularensis, and others that are a significant threat to human health.⁴⁹

In some embodiments, the pathogen contains a QseC homologue. The distribution of QseC in pathogens is discussed in references 17-19 and 50.

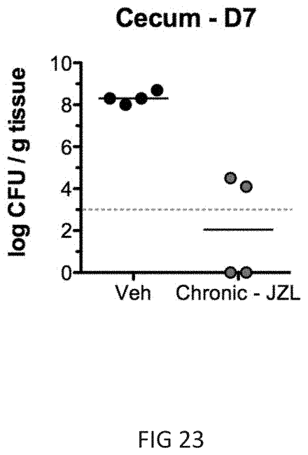

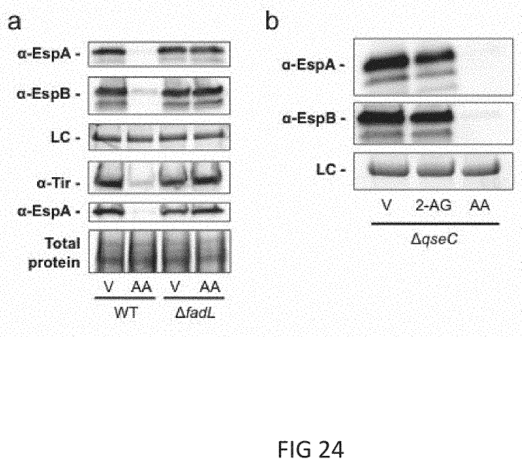

In some embodiments, the cannabinoid comprises 2-AG. In some embodiments, the cannabinoid comprises 2-AG, CBD, cannabidiolic acid (CBDA), cannabinol (CBN), cannabidivvarin (CBDV), cannabigerol (CBG), or a mixture thereof. In certain other embodiments, the cannabinoid comprises aracdonic acid (AA) or the MgI inhibitor JZL184. (See FIGS. 23 and 24.)

Pharmaceutical compositions or medicaments can be formulated by standard techniques or methods well-known in the art using one or more physiologically acceptable carriers or excipients. Suitable pharmaceutical carriers are described in, e.g., “Remington's Pharmaceutical Sciences” by E. W. Martin. Compounds and agents and their physiologically acceptable salts and solvates can be formulated for administration by any suitable route, including, but not limited to, oral, topical, nasal, rectal, by inhalation, or by injection (e.g., intravenous, subcutaneous, intramuscular, etc.), and combinations thereof. The therapeutic agent may be dissolved in a liquid, for example, in water or an organic solvent. The most suitable route of administration in any given case may depend in part on the nature, severity, and the stage of the disease. Co-administration of a plurality or combination of compounds may be by the same or different route of administration or together in the same pharmaceutical formulation.

In some embodiments, the pharmaceutical composition is administered in a plurality of doses. Such doses may be administered periodically (e.g., twice daily, daily, every other day, weekly) or as needed.

In certain embodiments, the subject is a human that has a gram negative bacterial infection. In other embodiments, the subject is a non-human animal that has such an infection. The non-human animal may be a domesticated animal, e.g., a livestock animal (e.g., a cow, sheep, goat, or pig) or other animal (e.g., a chicken, turkey, duck, salmon) that is being raised for human consumption or other use. Alternatively, the non-human animal may be a domesticated companion animal (dog, cat, horse, bird, reptile, fish, etc.). The non-human animal may also be a non-domesticated animal that has a gram negative bacterial infection.

In some embodiments, the subject may also be administered one or more antibiotics. The cannabinoid and the antibiotic may be administered in a single pharmaceutical composition or they may be administered separately. The antibiotic may be administered in a plurality of doses. Such doses may be administered periodically (e.g., twice daily, daily, every other day, weekly) or as needed.

In some embodiments, the methods for treating a subject having a gram negative bacterial infection further comprise the step of determining a characteristic of bacterial virulence. Bacterial virulence may be determined in one or more ways, including by evaluating the activity of the bacterial type 3 secretion system, the expression of a bacterial pathogenicity island, the secretion of bacterial effector proteins, lesion formation in the subject, and/or colonic tissue damage in the subject. This determination of bacterial virulence may be used to help guide the subject's treatment. For example, a medical or veterinary practitioner might prescribe additional doses of the pharmaceutical composition or adjust the amount of cannabinoid in the composition if bacterial virulence is greater than a threshold amount. Alternatively, the practitioner might add one or more antibiotics or other active pharmaceutical ingredients to the pharmaceutical composition.

The present disclosure also provides methods for prophylactically treating a subject at risk of developing a gram negative bacterial infection by administering to the subject an effective amount of a pharmaceutical composition comprising a cannabinoid and a pharmaceutically acceptable carrier.

In some embodiments, one or more aspects of the subject's condition may be evaluated after the administration of one or more prophylactic doses. In certain embodiments, bacterial virulence in the subject is determined at a time after the administration of the pharmaceutical composition. Further treatments may be administered, if appropriate. For example, the subject may be administered at least one additional dose of the pharmaceutical composition if bacterial virulence is greater than a threshold amount.

Such methods may be used for a variety of bacterial infections. In some embodiments, the methods may be used for an antibiotic-resistant infection. In a subset of such embodiments, the antibiotic-resistant infection is a gram negative bacterial infection. In some embodiments, the methods may be used for a subject with a GI tract infection. The infection may be with attaching and effacing bacteria. In some embodiments, the bacterial infection is an Enterobacteriaceae infection. In some embodiments, the bacterial infection is an infection by bacteria from a group of type III secretion system-encoding bacteria. In some embodiments, the gram negative bacterial infection is selected from at least one of Salmonella Typhimurium and enterohemorrhagic Escherichia coli infection.

In some embodiments, the present disclosure provides methods of reducing the ability of gram negative bacteria to invade and/or survive in a mammalian cell, e.g., an epithelial or immune cell, comprising administering to the cell a therapeutically effective amount of a pharmaceutical composition comprising a cannabinoid and a pharmaceutically acceptable carrier. In certain embodiments, the mammalian cell is a macrophage.

The present disclosure also provides methods for reducing bacterial virulence in a subject with a gram negative bacterial infection by administering a pharmaceutical composition comprising a cannabinoid and a pharmaceutically acceptable carrier to the subject. In certain embodiments, such methods further comprise determining a characteristic of bacterial virulence. Bacterial virulence may be determined using one or more methods including determining the activity of the bacterial type 3 secretion system, the expression of a bacterial pathogenicity island, the secretion of bacterial effector proteins, lesion formation, or colonic tissue damage.

The present disclosure further provides methods for managing bacterial virulence in a subject with a gram negative bacterial infection, comprising (i) performing an initial measurement of bacterial virulence in the subject, (ii) administering to the subject at least one dose of a pharmaceutical composition comprising a cannabinoid and a pharmaceutically acceptable carrier if the initial measurement of bacterial virulence is greater than a threshold amount, (iii) performing at least one later measurement of bacterial virulence in the subject at a time period after the most recent administration of the pharmaceutical composition, and (iv) administering to the subject at least one additional dose of the pharmaceutical composition if the later measurement of bacterial virulence is greater than a threshold amount. The subject may also be monitored for other criteria, e.g., expression of various cytokine genes. In certain embodiments, the pharmaceutical composition also comprises one or more antibiotics.

The present disclosure also provides methods for reducing bacterial virulence in a subject having an antibiotic-resistant gram negative bacterial infection, comprising administering to the subject a therapeutically effective amount of a pharmaceutical composition comprising a cannabinoid and a pharmaceutically acceptable carrier to reduce a pathological effect or symptom of the antibiotic-resistant gram negative bacterial infection. In certain embodiments, the bacterial infection is multidrug resistant. In some embodiments, the bacterial infection may be a Carbapenem-resistant Enterobacteriaceae (CRE) infection, an extended-spectrum Beta-lactamase producing Enterobacteriaceae infection, a drug-resistant non-typhoidal Salmonella infection, a drug-resistant Salmonella Serotype Typhi infection, or a drug-resistant Shigella infection.

The pharmaceutical composition may be as previously described. This composition may be dosed as previously described.

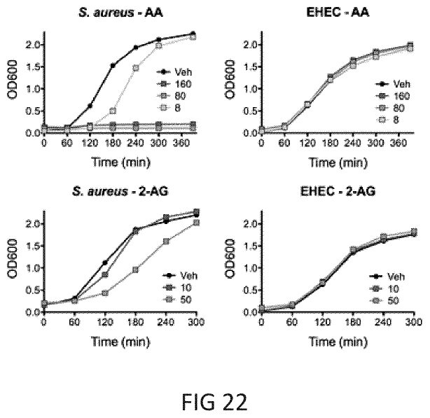

2AG does not act by preventing bacterial growth or killing bacteria. Some lipids are antimicrobial with respect to certain bacteria. Arachidonic acid, for example, inhibits the growth of certain gram-positive species such as S. aureus. By contrast, 2AG does not inhibit growth of gram-negative bacteria such EHEC, even at concentrations of 80 and 160 μM (FIG. 22).

The results described in this disclosure represent the first description of a bacterial signaling node within the host endocannabinoid system. It was not previously known that 2AG could be sensed by bacteria, activate intracellular signaling cascades, and modulate bacterial function. We show that at physiological levels of 10 μM, which have been reported for 2AG to signal mammalian cells in the gut, 2AG is also serving as a signal to bacteria. This disclosure builds on this information and provides important new methods for the benefit of human and animal health.

### EXAMPLES

To further illustrate these embodiments, the following examples are provided. These examples are not intended to limit the scope of the claimed invention, which should be determined solely on the basis of the attached claims.

### Example 1—Mice with Elevated 2-AG Levels are Resistant to Infectious Colitis

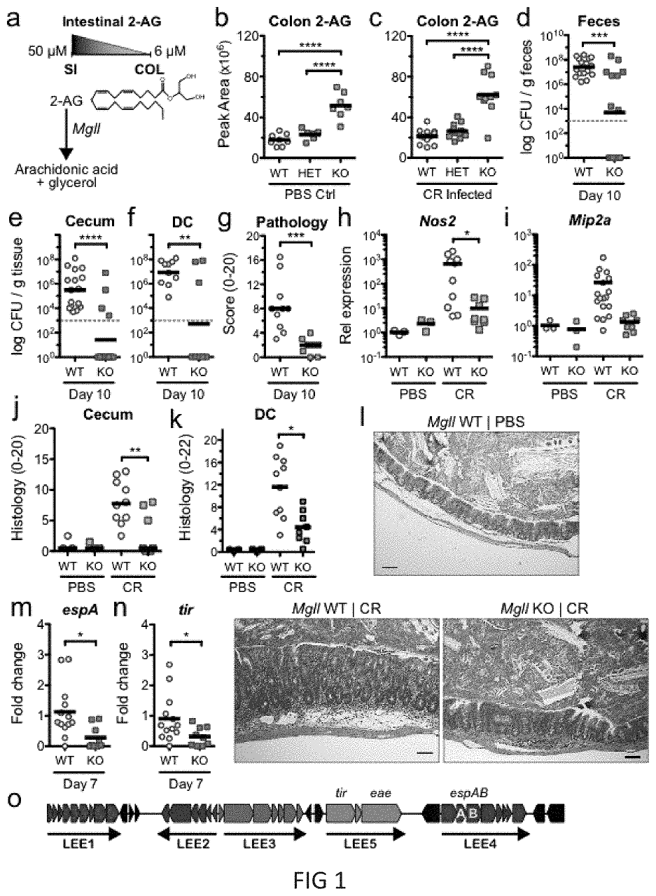

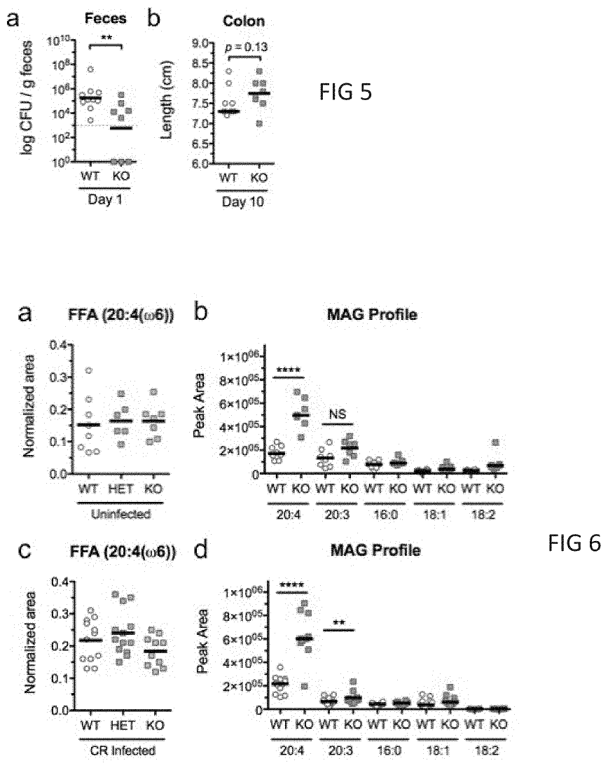

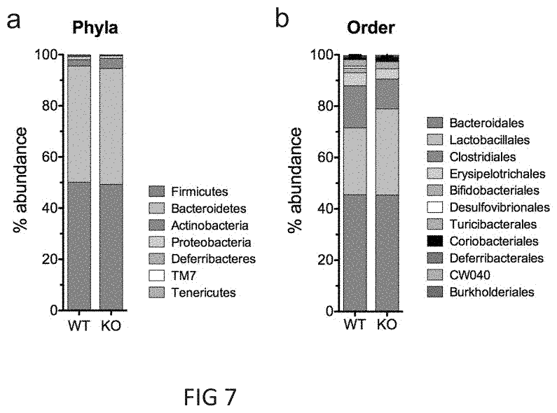

Inactivation of the host 2-AG hydrolytic enzyme, monoacylglycerol lipase (MagI), increases 2-AG levels in various organs including the colon (FIG. 1a-c).^{7,8} To initially establish whether endocannabinoids alter host susceptibility to bacterial infection, MagI deficient (MgII KO) mice and MagI sufficient (MgII WT) littermate controls were infected with the murine attaching and effacing (A/E) pathogen Citrobacter rodentium (FIG. 1d-l). Pathogen burdens in stools and in colonic tissues were significantly decreased in MgII KO mice relative to MgII WT controls (FIG. 1d-f, FIG. 5a). This corresponded with attenuated colonic tissue damage, histopathology and decreased expression of various proinflammatory genes (FIG. 1g-l, FIG. 5b). Inactivation of MagI did not alter colonic arachidonic acid levels, which is a breakdown product of 2-AG hydrolysis (FIG. 1a, FIG. 6). Moreover, colonic concentrations of other monoacylglycerols (MAG) remained unchanged in MgII KO mice (FIG. 6), demonstrating the specificity of MgII deficiency in altering 2-AG levels in the gut. Finally, the composition of the microbiotas of MgII WT versus KO mice were comparable prior to infection, suggesting that the differences in infection response is unlikely mediated by the microbiota (FIG. 7). Taken together, mice with elevated levels of 2-AG are more resistant to C. rodentium infection.

A/E pathogens such as C. rodentium, enterohemorrhagic Escherichia coli (EHEC) and enteropathogenic E. coli (EPEC) harbor the locus of enterocyte effacement (LEE) pathogenicity island that is essential for successful intestinal colonization and consequent disease induction (FIG. 10). The LEE encodes a type 3 secretion system (T3SS), a molecular syringe-like structure that injects bacterial proteins known as effectors into epithelial cells to establish a replicative niche within the intestines.⁹ Genetically engineered mutants that can no longer assemble this molecular machine and/or translocate key effectors into host cells are unable to form A/E lesions on epithelial cells or colonize the intestines.^{10,9} We therefore determined whether attenuation of colitis in C. rodentium-infected MgII KO mice corresponds with decreased activation of the LEE. In MgII KO mice, C. rodentium recovered from cecal contents exhibited decreased expression of the espA gene that encodes the translocon component and the gene that encodes the effector Tir, both of which are essential for A/E lesion formation on enterocytes and disease^{9,10} (FIG. 1m-n). Thus, full activation of the LEE in C. rodentium is inhibited in mice with elevated levels of 2-AG.

Pharmacological inhibition of MgI with the with the MgI inhibitor JZL184 decreases colonization of cecum tissues by C. rodentium (FIG. 23).

### Example 2—2-AG Inhibits LEE-Encoded T3SS Activity in A/E Pathogens

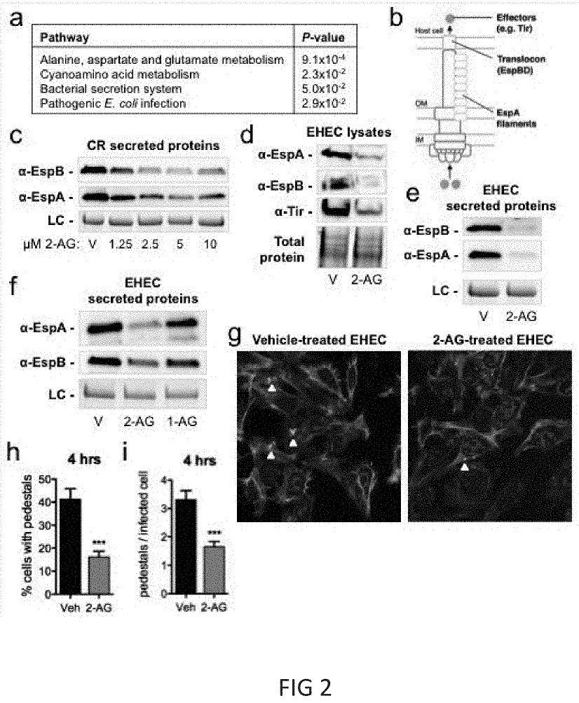

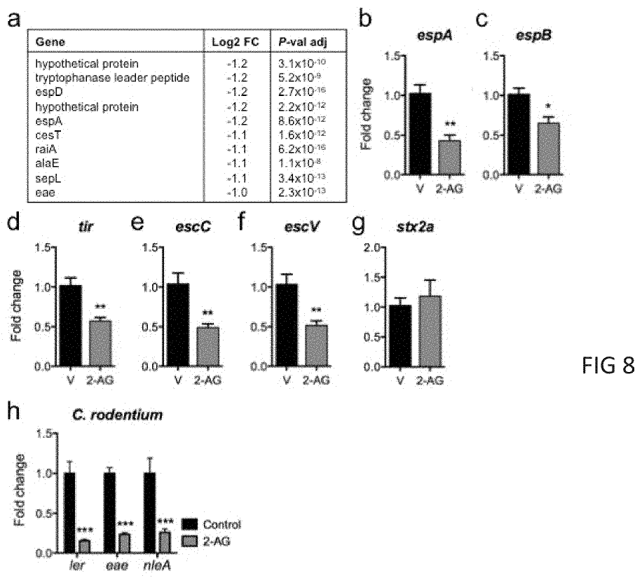

We next investigated whether 2-AG directly inhibits LEE expression and activity in C. rodentium and the human A/E pathogen EHEC. To accomplish this, we cultivated EHEC under LEE-inducing conditions in the presence of 10 μM 2-AG, which is within the physiological range reported in human and murine intestines.^{7,11} We then performed RNAseq to investigate whether 2-AG alters the pathogen transcriptome (RNAseq accession number PRJEB29880). In comparison to vehicle-treated cultures, 2-AG significantly altered the expression four KEGG pathways (FIG. 2a), including the bacterial secretion system and pathogenic E. coli infection pathways, which corresponded with significant downregulation of numerous LEE-encoded genes (FIG. 8a). The inhibitory effects of 2-AG on LEE transcription were confirmed by targeted qPCR of LEE-encoded genes spanning its five operons (FIG. 8b-f). Similarly, 2-AG treatment corresponded with decreased expression of LEE-encoded genes in C. rodentium (FIG. 8h). Interestingly, 2-AG had no transcriptional effect on Shiga toxin (stx2a), a lethal virulence factor in EHEC (FIG. 8g). Moreover, few non-LEE encoded genes were differentially expressed in EHEC in response to 2-AG (RNAseq accession number PRJEB29880). Taken together, the inhibitory effects of 2-AG on A/E pathogens seem to target the LEE.

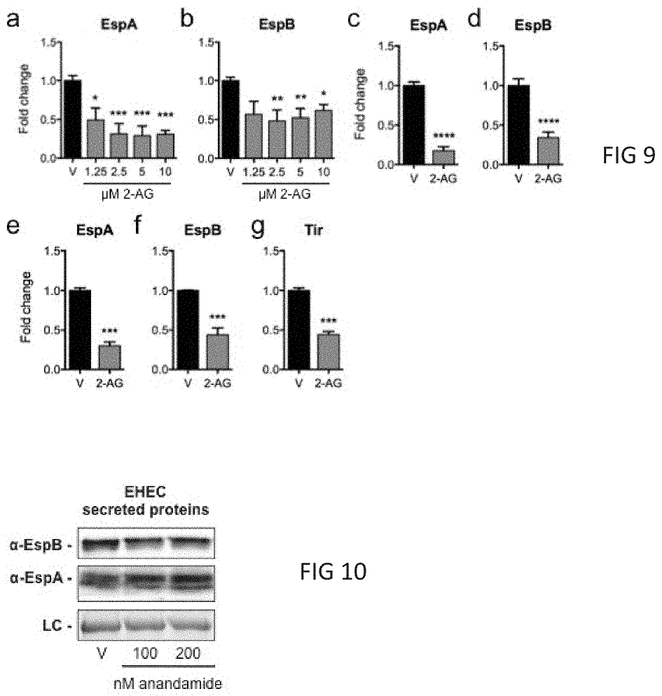

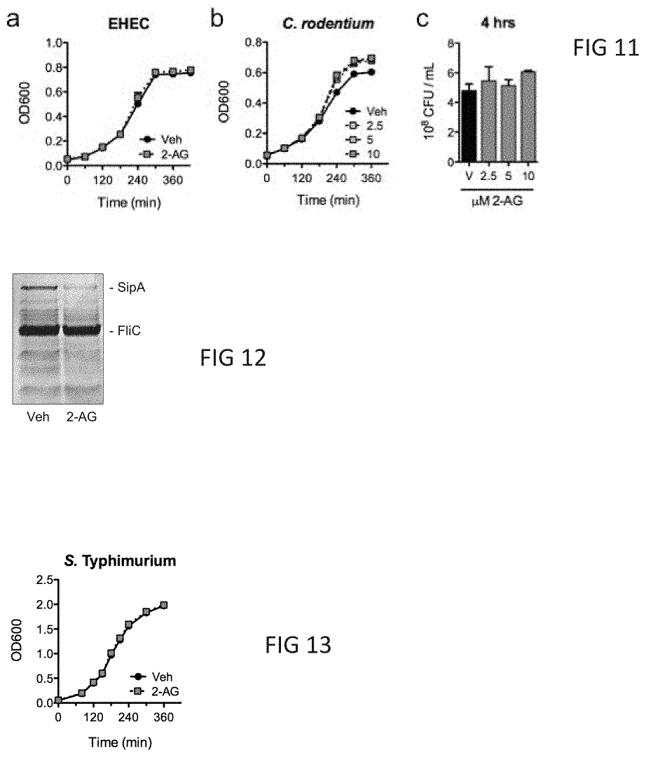

To functionally confirm the inhibitory effects of 2-AG on the LEE, we next investigated whether 2-AG modulates LEE-encoded T3SS activity. We cultivated C. rodentium or EHEC with physiological concentrations of 2-AG and assessed secretion of two translocon components, EspA and EspB (FIG. 2b). The presence of 2-AG significantly reduced EspA and EspB secretion (FIG. 2c, 2e, FIG. 9a-d). Similarly, protein expression of EspA, EspB and Tir was significantly decreased in EHEC when exposed to 2-AG (FIG. 2d, FIG. 9e-g). The biological activity of 2-AG can be inactivated through isomerization into 1-AG.¹² In contrast to 2-AG, the 1-AG isoform does not attenuate LEE-encoded T3SS activity (FIG. 2f). Moreover, the endocannabinoid anandamide, which also contains an arachidonic acid moiety and is abundant in the gut, does not alter LEE activity (FIG. 10), demonstrating the specificity of the effects of 2-AG on A/E pathogens. Importantly, 2-AG inhibition of the LEE did not correspond with altered growth (FIG. 11). Hence 2-AG does not act as an antimicrobial, but as an anti-virulence signal. We next determined whether 2-AG also attenuates LEE-dependent epithelial A/E lesion formation by EHEC (i.e. pedestal formation) (FIG. 2g, arrowheads). 2-AG-treated EHEC exhibited decreased pedestal formation on epithelial cells (FIG. 2g-i), further demonstrating the inhibitory effects of 2-AG on the virulence potential of A/E pathogens.

### Example 3—2-AG Inhibition of Pathogenic T3SS Activity is Conserved in S. Typhimurium

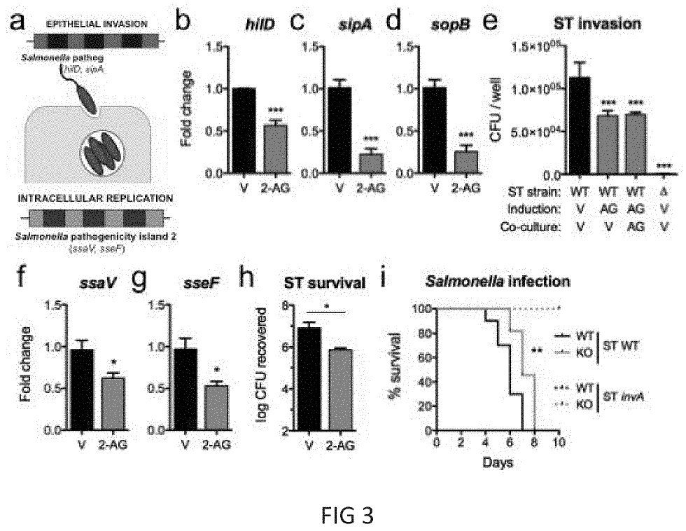

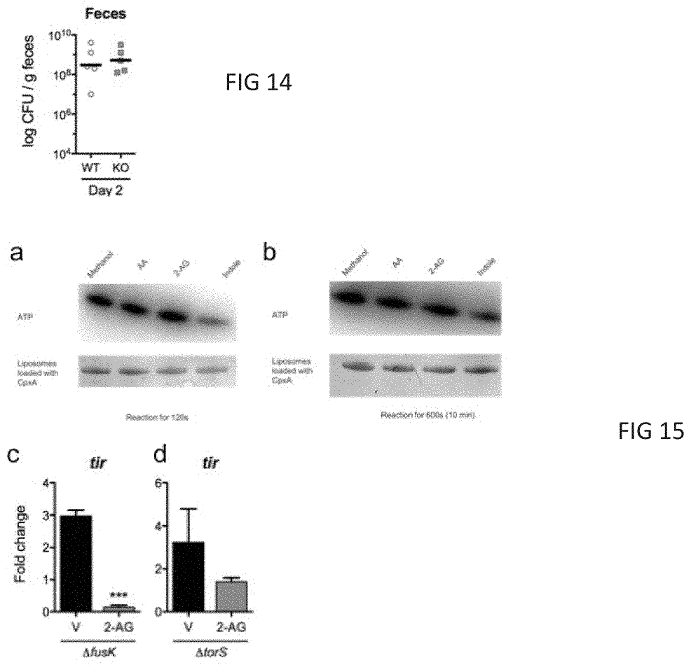

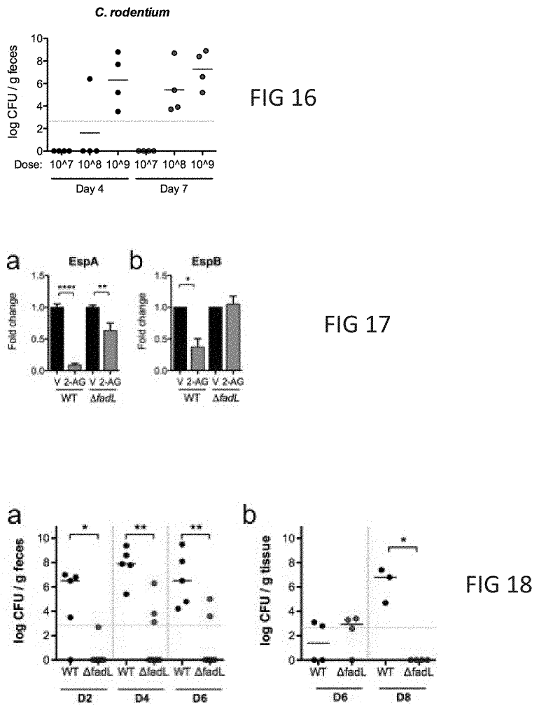

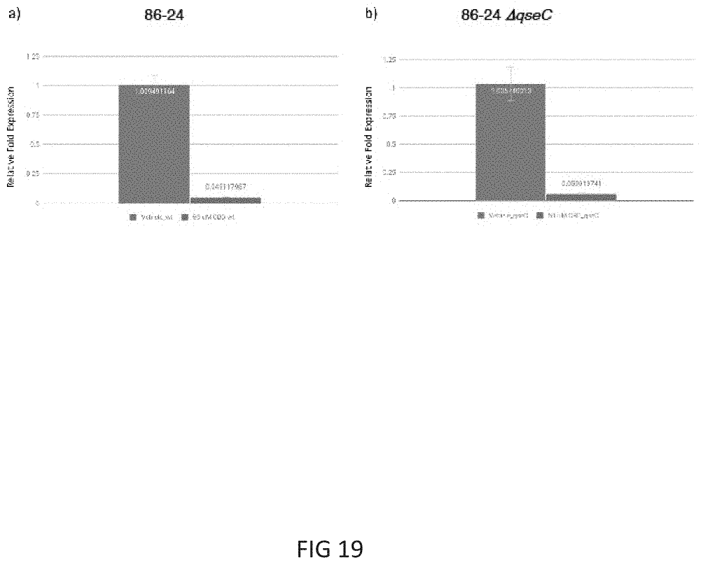

To establish whether the anti-virulence effects of endocannabinoids are conserved in other Enterobacteriaceae, we investigated whether 2-AG modulates the virulence potential of the invasive enteric pathogen Salmonella enterica serovar Typhimurium. S. Typhimurium harbors the Salmonella pathogenicity islands 1 (SPI-1) and SPI-2, which each encode T3SSs that confer epithelial invasiveness and the ability to replicate within intracellular environments respectively (FIG. 3a).^{13,14} To determine whether endocannabinoids modulate SPI-1 activity, we cultured S. Typhimurium under SPI-1 inducing conditions in the presence of 2-AG. Targeted qPCR of SPI-1 genes revealed that 2-AG inhibits expression of the transcriptional regulator hilD and the SPI-1 effectors sipA and sopB (FIG. 3b-d), which corresponded with decreased secretion of SipA (FIG. 12) and attenuated epithelial invasiveness (FIG. 3e). Importantly, 2-AG did not alter S. Typhimurium growth kinetics (FIG. 13). We cultured S. Typhimurium under SPI-2 inducing conditions to determine whether 2-AG also modulates SPI-2 activity. Exposure to 2-AG resulted in decreased expression of the SPI-2-encoded T3SS apparatus protein ssaV and effector sseF (FIG. 3f-g),¹⁵ and decreased survival within macrophages (FIG. 3h). To assess whether 2-AG attenuates SPI-1-mediated virulence in vivo, we infected streptomycin-treated mice with S. Typhimurium WT or a mutant with inactivated SPI-1 activity (ΔinvA). MgII KO mice exhibited significant protection against S. Typhimurium infection as assessed by survival (FIG. 3i), despite similar fecal burdens of S. Typhimurium (FIG. 14). Taken together, 2-AG inhibits two pathogenicity islands in S. Typhimurium that are essential for establishing successful infection in the host, therefore demonstrating the broader anti-virulence effects of endocannabinoids on enteric pathogens.

### Example 4—2-AG Mediates its Anti-Virulence Effects by Counteracting Activation of the Bacterial Adrenergic Receptor QseC

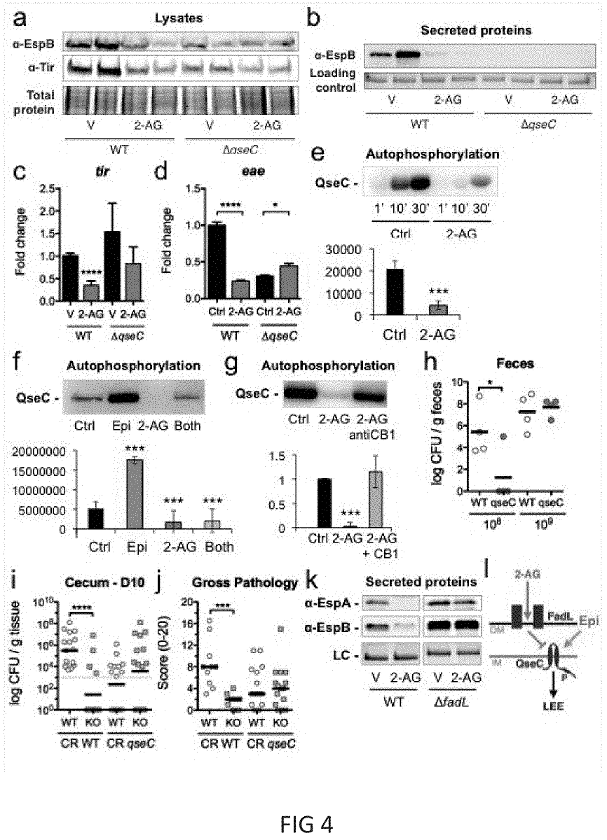

In pathogens and pathobionts, including EHEC, C. rodentium, S. Typhimurium, and adherent-invasive E. coli, the host peptide-based neurotransmitters epinephrine (epi) and norepinephrine (NE) promote intestinal colonization and consequent disease through positive regulation of associated virulence programs.¹⁶⁻²⁰ Epi/NE are sensed by bacterial cells through the adrenergic receptor QseC, a histidine kinase (HK) that integrates these chemical cues into intracellular signaling cascades that modulates bacterial physiology, behavior and function.¹⁶ In EHEC and C. rodentium, QseC activation enhances LEE-encoded T3SS activity (FIG. 4a-b).^{16,19} Similarly, Epi/NE generally serve as positive signals in mammalian physiology, which are counteracted by the inhibitory effects of endocannabinoids.²¹ We therefore hypothesized that 2-AG may also inhibit Epi/NE-mediated activation of the LEE as its mechanism of action. To test this, we first assessed whether LEE activity is also inhibited by 2-AG in a qseC isogenic mutant. In contrast to the parental strain, LEE functional activity in the qseC mutant in EHEC was not affected by 2-AG (FIG. 4a-b). Together, these data suggest that QseC may also be critical for 2-AG sensing in bacteria.

We next investigated whether 2-AG directly interacts with QseC to mediate its inhibitory effects. To accomplish this, purified QseC was reconstituted into liposomes and exposed to 2-AG to assess its effects on QseC autophosphorylation, an indicator of HK activity. QseC activation was significantly diminished in the presence of 2-AG (FIG. 4c). Moreover, 2-AG inhibited Epi-mediated activation of QseC (FIG. 4d). To further demonstrate that 2-AG specifically engages with QseC, we introduced the inhibitor AM251, which counteracts endocannabinoid signaling by antagonizing their cognate receptor cannabinoid receptor 1 (CBR1). As in mammalian cells, the CBR1 antagonist prevented 2-AG-mediated inhibition of QseC activity in an in vitro liposome system (FIG. 4e), which further supports our hypothesis that 2-AG can interact with QseC. Importantly, 2-AG did not inhibit the activities of other non-adrenergic HKs including CpxA and FusK (FIG. 15), suggesting specificity of 2-AG signaling through QseC.

The contribution of QseC to the virulence potential of C. rodentium is dependent on the initial infectious dose in susceptible C3H/HeJ mice.¹⁹ Because the MgII heterozygous and homozygous deficient mice are on the C57131/6 background, we determined the minimum infectious dose of C. rodentium required to establish successful colonization and concluded that at least 10⁸ colony forming units (CFU) was necessary (FIG. 15). Similar experiments with the qseC mutant revealed that at least 10⁹ CFUs were required for intestinal colonization (FIG. 4f). To demonstrate in vivo relevance of QseC-mediated pathogen sensing of 2-AG, we challenged MgII KO mice with elevated 2-AG levels and MgII WT littermate controls with 10⁹ CFUs of C. rodentium WT or the qseC mutant. In contrast to infection with the parental strain, MgII KO mice challenged with the qseC mutant no longer exhibited protection against C. rodentium infection as assessed by intestinal pathogen burden and gross pathology (FIG. 4g-i). However, as expected, cecum colonization and gross pathology in both murine strains is attenuated in animals infected with the qseC mutant compared to WT (FIG. 4g-i). The minimal differences in CFU in stools observed between the parental and qseC mutant have been previously reported for this high infectious dose.¹⁹ Collectively, these data demonstrate that 2-AG acts as a host-derived QseC antagonist and counteracts QseC-mediated activation of the LEE in A/E pathogens.

QseC is a transmembrane protein localized to the inner membrane of bacterial cells,²² which introduces the question of how 2-AG crosses the outer membrane to engage with this HK. The structure of 2-AG is comprised of an arachidonic acid moiety covalently bonded to a glycerol backbone at the sn2 position (FIG. 1a).²³ In Enterobacteriaceae, FadL is the only identified long chain fatty acid transporter.²⁴ We therefore hypothesized that FadL may also serve as a 2-AG transporter in Enterobacteriaceae. LEE activity was no longer inhibited in an isogenic fadL mutant in response to 2-AG (FIG. 4I, FIG. 17), suggesting that FadL is essential for pathogen sensing of 2-AG. We attempted to investigate how mice with elevated 2-AG levels respond to infection of the C. rodentium fadL mutant. However, deletion of fadL rendered C. rodentium unable to stably colonize the murine gut (FIG. 18).

These findings establish that Enterobacteriaceae pathogens respond transcriptionally, behaviorally and physiologically to host-derived endocannabinoids. Moreover, the endocannabinoid 2-AG serves as an inhibitory signal that dampens the virulence potential of enteric pathogens. These results support a model of endocannabinoid signaling in A/E pathogens that depends on initial transport through FadL and subsequent inhibition of the bacterial adrenergic receptor QseC, resulting in attenuated downstream activation of the LEE (FIG. 4m). Because QseC and FadL are encoded in the core genomes of Enterobacteriaceae, endocannabinoids may also modulate additional aspects of bacterial function in both pathogenic and commensal bacteria. More broadly, these findings introduce the possibility that intestinal bacteria may serve as an additional and potentially druggable signaling node within the enteric endocannabinoid system that impacts GI physiology, immunity and susceptibility to infection.

### Example 5—CBD Reduced the Expression of EHEC espA

Based on reduced expression of type III secretion system components and virulence in a mouse infection model in the presence of 2-AG, we investigated the effects of the related compound cannabidiol (CBD) for similar phenotypes. We grew EHEC strain 86-24 in low glucose DMEM to mid-exponential phase in the presence and absence of 50 μM CBD (Enjoi CBD solution, Khaleafa Cannabis Company, Portland, Oreg.). Whole cell RNA was harvested and qPCR performed, using rpoA as an internal control for Ct analysis. Previous results indicated that 2-AG signaling was dependent upon the quorum sensing regulator QseC, so we also interrogated the regulation using the ΔqseC deletion strain, isogenic to wt EHEC strain 86-24.

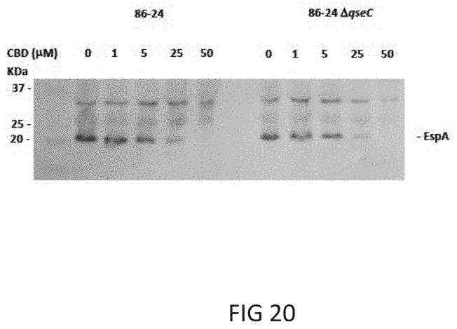

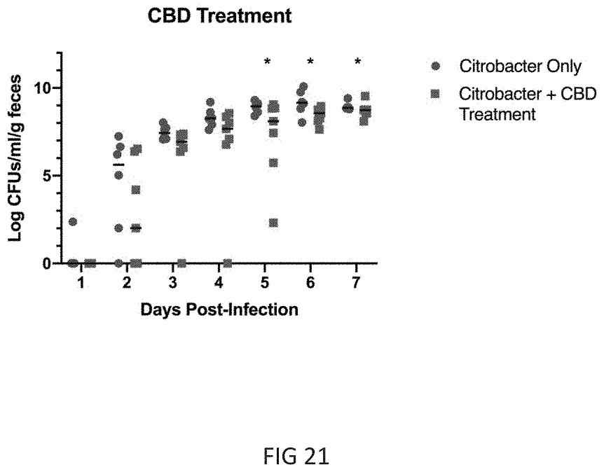

We observed a 20.6-fold decrease in expression of espA in the presence of CBD compared to the vehicle (FIG. 20). In the case of the ΔqseC deletion strain, we observed a 17.5-fold reduction in expression of espA between the control and CBD; this effect was not qseC-dependent. We also monitored expression of EspA protein using immunoblot, assayed under the same conditions. Consistently, we observed reduced expression of EspA in the presence of 50 μM CBD (FIG. 21). As a control, we determined that neither the growth rate nor the growth yield for the wt and ΔqseC deletion strains were significantly altered in the presence of 20 μM or 50 μM CBD compared to the vehicle control. We concluded that the presence of 50 μM CBD significantly diminished the expression of espA in a qseC-independent manner.

### Example 6—Post-Infection CBD Treatment Reduced Citrobacter rodentium Colonization in a Mouse Model

To examine the in vivo efficacy of CBD as a potential therapeutic against Gram-negative, type III secretion system-containing pathogens, we used the mouse pathogen C. rodentium. We examined infection through fecal load using colony count techniques, selecting on nalidixic acid plates to distinguish the pathogen from the normal fecal microbiota. Two groups of mice were infected from the same culture, and this approach was then repeated in a second, independent assay. One of the two treatment groups were orally administered CBD 1-day post infection and standard plate counts were examined over the course of the assay. Colony counts were all normalized for weight of feces. Data obtained from each mouse were logarithmically-scaled and averaged (FIG. 22). Biological replicates were utilized for analysis through the Mann-U Whitney model. Using a two-tailed alpha value of 0.05, it was found that for days five-seven post-infection, the vehicle and orally administered CBD treatment groups were statistically significantly different from one another.

Extending on the work showing that the endocannabinoid 2-AG reduces expression of type III secretion system components in multiple Gram-negative pathogens, including EHEC and Salmonella enterica, and overproduction of 2-AG in a recombinant mouse diminishes virulence using the mouse pathogen C. rodentium, we demonstrate similar results for the related exogenous cannabinoid, cannabidiol, or CBD. The commercially available CBD product diminished expression of the type III component EspA in EHEC, and reduced colonization of C. rodentium in the mouse model of infection. Using an established cannabis reference laboratory, we established that the ratio of CBD:THC was 10:1, typical for CBD purification from marijuana. In sum, this work offers an empirical approach to the discovery of CBD as a nontoxic, readily available compound to act as a broad-spectrum, quorum sensing inhibitor directed against a diverse range of Gram-negative pathogens.

Materials and Methods:

Bacterial strains and growth conditions. Bacterial strains used in this study are listed in Table 1. Isogenic mutants were generated using the lamda red recombinase method as previously described.²⁵ Unless otherwise indicated, all bacterial strains were grown overnight aerobically in LB with antibiotics for maintenance of plasmids as appropriate prior to subculture for in vitro experiments or mouse infections. E. coli and C. rodentium strains were subcultured in DMEM with 0.1% glucose (Gibco) and grown in standing cultures (microaerobic) or shaking at 250 rpm (aerobic) at 37° C. (LEE-inducing conditions). S. Typhimurium strains were subcultured in LB (SPI-1-inducing conditions) or subcultured in N9 minimal medium (SPI-2-inducing conditions) and grown aerobically at 37° C. Where indicated, bacteria were grown in the presence of 2-AG (Tocris) or the vehicle control (methanol, final concentration of 1:10000).

TABLE 1
Bacterial strains used in this study

| Strains | Description | Reference |
|---|---|---|
| 86-24 | Shigatoxin-positive EHEC | (Griffin P M 1988) |
| | strain serotype O157:H7, | |
| | streptomycin resistant | |
| VS138 | qseC isogenic mutant in 86-24 | 33 |
| 86-24 fadL | fadL isogenic mutant in 86-24 | 34 |
| 86-24 qseE | qseE isogenic mutant in 86-24 | 35 |
| 86-24 fusK | fusK isogenic mutant in 86-24 | 36 |
| mCherry-expressing | 86-24 transformed with | This study |
| 86-24 | pDP151 | |
| SL1344 | Reference Salmonella enterica | 37 |
| | serovar Typhimurium strain | |
| SL1344 invA spiB | invA spiB isogenic mutant in | 38 |
| | SL1344 | |
| IR715 | Reference Salmonella enterica | 43 |
| | serovar Typhimurium strain | |
| IR715 invA | invA spiB isogenic mutant in | 43 |
| | IR715 | |
| DBS100 | Reference Citrobacter | 39 |
| | rodentium strain Nalidixic | |
| | acid resistant | |
| DBS100 qseC | qseC isogenic mutant in | 40 |
| | DBS100 | |

Overnight cultures of wild type EHEC 86-24 or the deletion strain, ΔqseC, were prepared in 3 ml LB medium, then placed overnight in the 37° C. incubator, shaking at 250 rpm. The following day the overnight cultures were vortexed and pipetted (1:100) into sterile filtered room-temperature Low Glucose (0.1%)-Dulbecco's Modified Eagle Medium (LG-DMEM). The LG-DMEM (Sigma D2902-1L) was prepared using the powder, with added 3.7 g NaHCO3 (60.63 mM), and MilliQ water to adjust to the final 1 L volume (pH 7.0). Prior to overnight inoculation, the room temperature LG-DMEM was dispensed into 3 ml aliquots supplemented with 30 μL of 10% methanol (Vehicle), 23 μL (20 μM) of room temperature Enjoi CBD solution (Khaleafa Cannabis Company, Portland, Oreg.), or 58 μL (50 μM) of room temperature Enjoi CBD solution. The CBD solution was stored at 4° C. in a low-light environment to prevent any photolytic degradation and thoroughly mixed before being added to the LG-DMEM. Standing cultures were then placed in a 37° C. incubator without shaking for 5 hours. At the 5-hour mark the cultures were removed from the incubator and the optical densities (OD600) were measured, blanked with LG-DMEM. The optical densities were recorded and the cultures were used for RNA purification. Cultures were grown in an identical manner, and then whole cell proteins harvested for immunoblot analysis.

Mice. C57BL/6 mice were purchased from The Jackson Laboratory. MgII^{+/-} breeding pairs were recovered from cryopreservation (Texas A&M Institute for Genomic Medicine, College Station, Tex.) and utilized to generate a mouse colony at UT Southwestern. Genotypes were confirmed using the following primer pairs: MgII 5′ F7: GGAAACAGGTTTGTCATGGC (SEQ ID NO:1), and MgII 3′ R1: GCGAGAAACCAGAAGGAGAC (SEQ ID NO:2). All mice used in Examples 1-4 were housed in specific pathogen-free conditions at UT Southwestern. All animal protocols were approved by either the UT Southwestern Medical Center Institutional Animal Care and Use Committee or OHSU's Department of Comparative Medicine in accordance with IACUC regulation.

C. rodentium infections. At 8-12 weeks of age, male and female WT, MgII^{+/-} or MgII^{-/-} mice on the C57BL/6 background or C57BL/6 mice from Jackson Laboratory were orally infected with 1×10⁹ CFU of C. rodentium DBS100 or mock infected with PBS. Fecal pellets were collected throughout the course of infection to enumerate fecal pathogen loads by quantitative culture using selective media with nalidixic acid. At necropsy, cecal tissues were harvested to quantify C. rodentium burden, for pathology analysis and for RNA isolation from tissues. Cecal contents were also collected for RNA isolation.

S. enterica serovar Typhimurium infections. At 8-12 weeks of age, male and female WT or Mg^{-/-} mice on the C57BL/6 background were intragastrically administered 20 mg of streptomycin as previously described.⁴³ After 24 hours, mice were orally infected with 1×10⁹ CFU of S. enterica Typhimurium IR715 WT or the SPI-1-deficient mutant, ΔinvA. Fecal pellets were collected throughout the course of infection to enumerate fecal pathogen loads by quantitative culture using selective media with nalidixic acid. Severity of disease was assessed by mouse survival.

RNA isolation and quantitative real-time PCR. RNA was isolated from cecal tissues snap frozen in liquid nitrogen using the TriZol method (Thermo Fisher Scientific) according to the manufacturer's instructions. For bacterial expression of LEE genes in vivo, RNA was isolated from cecal contents snap frozen in liquid nitrogen using the RNeasy PowerMicrobiome Kit (Qiagen) according to the manufacturer's instructions. For in vitro bacterial RNA isolations, all strains were grown to mid-log or late-log phase as indicated and bacterial RNA was extracted using the RiboPure bacterial isolation kit (Ambion) per the manufacturer's instructions. cDNA was synthesized using SuperScript II reverse transcriptase (ThermoFisher Scientific). qPCR was performed in a QuantStudio 6 Flex Instrument (Life Technologies) with Power SYBR Green (Applied Biosystems) using the following PCR conditions: a single hold at 50° C. for 2 minutes and at 95° C. for 10 minutes, followed by 40 cycles at 95° C. for 15 seconds and 60° C. for 1 minute. Each PCR was performed in 10 μL reactions and contained the following: 1×SYBR green mix and 0.25 μM of each primer. Melting curves were assessed to ensure specificity of the PCR products. The following primers were utilized to amplify mammalian transcripts: Gapdh—FP-GGTGAAGGTCGGAGTCAACGGA (SEQ ID NO:3); RP-GAGGGATCTCGCTCCTGGAAGA (SEQ ID NO:4); Nos2—FP-TTGGGTCTTGTTCACTCCACGG (SEQ ID NO:5), RP-CCTCTTTCAGGTCACTTTGGTAGG (SEQ ID NO:6).²⁶ The relative abundance of mammalian mRNA transcripts was calculated using the delta delta CT method and normalized to Gapdh levels. Table 2 lists qRT-PCR primers to amplify bacterial transcripts. The relative abundance of bacterial mRNA transcripts was calculated using the delta delta CT method and normalized to rpoA levels.

TABLE 2
Oligonucleotide primers used in this study

| Name | Sequence (5′ to 3′) | Reference |
|---|---|---|
| EHEC espA | FP-AGCTATTTGAGGAACTCGGTG (SEQ ID NO: 7) | This work |
| | RP-CATCTTTTGTGCCGTGGTTG (SEQ ID NO: 8) | |
| EHEC espB | FP-GGTCAAGGCTACGGAAAGTG (SEQ ID NO: 9) | This work |
| | RP-TCTTCAGCAAAGTCAGAGGC (SEQ ID NO: 10) | |
| EHEC tir | FP-GAGGGAGTCAAATAGCGGTG (SEQ ID NO: 11) | This work |
| | RP-ATCTGAACGAAGGCTGGAAG (SEQ ID NO: 12) | |
| EHEC escC | FP-CTGAAGACAATGGCAAGTAATGG (SEQ ID NO: 13) | |
| | RP-ACTGCATTAAGACGTGGATCAG (SEQ ID NO: 14) | This work |
| EHEC escV | FP-GAGTGCAAAAGGAAAGCCAG (SEQ ID NO: 15) | This work |
| | RP-ATGATACCAGCAATAGCGTCC (SEQ ID NO: 16) | |
| EHEC rpoA | FP-GTGACCCTTGAGCCTTTAGAG (SEQ ID NO: 17) | This work |
| | RP-ACACCATCAATCTCAACCTCG (SEQ ID NO: 18) | |
| ST hilD | FP-GAGATACCGACGCAACGACT (SEQ ID NO: 19) | This work |
| | RP-TTGGTTTGCTGCTCGTTTGG (SEQ ID NO: 20) | |
| ST sipA | FP-ATTGCACTGCAGTTTGCCAG (SEQ ID NO: 21) | This work |
| | RP-GGTATGACTCGTAAGCCGGG (SEQ ID NO: 22) | |
| ST sopB | FP-CGGGTACCGCGTCAATTTC (SEQ ID NO: 23) | 31 |
| | RP-TGGCGGCGAACCCTATAAA (SEQ ID NO: 24) | |
| CR_rpoA | FP-ACGTCAGCCGGAAGTGAAAGAAGA (SEQ ID NO: 25) | This work |
| | RP-AGCGGACAGTCAATTCCAGATCGT (SEQ ID NO: 26) | |
| CR_espA | FP-TGACAACGACGGTGGATAATAG (SEQ ID NO: 27) | This work |
| | RP-GCCAATGGGTATTGCTGAAAC (SEQ ID NO: 28) | |
| CR_tir | FP-ATCAGATATCTCGCAAGCTCG (SEQ ID NO: 29) | This work |
| | RP-CAACTCCATCTCCCATTCCTG (SEQ ID NO: 30) | |

Gross pathology and histopathology. At necropsy, colitis severity was first grossly assessed, including colon lengths and qualitative assessment of cecal atrophy (0-4), thickening of cecal (0-5) and colon tissues (0-4), extent of content loss in the cecum (0-4) and diarrhea (0-3). Total gross pathology scores is on a scale from 0-20. For histopathology assessment, the cecal tip and Swiss rolled distal colonic segments were fixed in 10% neutral buffered formalin. Histological inflammation scores (0-23) of hematoxylin and eosin stained colonic sections were blindly assessed as previously described.²⁷ Briefly, inflammation was assessed based on the following histopathological features: submucosal edema (0-4), goblet cell depletion (0-4), epithelial hyperplasia (0-4), epithelial integrity (0-4), polymononuclear (PMN) cell and inflammatory monocyte infiltration (0-4) and bacterial epithelial attachment (0-3). Data are expressed as the sum of these individual scores (0-23).

RNAseq library preparation, sequencing and analysis. RNA was isolated from four biological replicates and sequenced at the UT Southwestern Medical Center Next Generation Sequencing Core. RNA libraries were prepared using Illumina ScriptSeq Complete Kit (Bacteria) (Catalog #BB1224). RNA libraries were run on the Illumina HiSeq 2500 sequencer with SE-50. To analyze the data, reads were trimmed, decontaminated and quality-filtered using BBmap software suite. Reads were mapped to the Escherichia coli O157: H7 str. EDL933 genome using Bowtie2. Number of reads of each gene was determined using the featureCounts package and differential expression was analyzed using DESeq2. Accession number is PRJEB29880.

Western blot assays for lysate-associated and secreted proteins. Secreted proteins were isolated at the indicated time points as previously described.²⁸ Bovine serum albumin (BSA) was used as a loading control and added to secreted protein samples. Cell pellets were resuspended in 8M urea to harvest lysate-associated proteins. Proteins were separated by a gradient 4-15% SDS-PAGE gel, transferred to a polyvinylidene fluoride membrane and blocked with 5% milk or 5% BSA (as appropriate) in phosphate buffered saline (PBS) with 0.05% Tween. Membranes were probed with anti-EspA, anti-EspB or anti-Tir primary antibodies, followed by incubation with secondary antibodies conjugated to streptavidin-horseradish peroxidase. Membranes were exposed with the Bio-Rad ChemiDoc Touch Imaging System with Image Lab 5.2.1 software for image analysis.

Fluorescent actin staining (FAS) assay. FAS assays were performed on HeLa cells as previously described.²⁹ Briefly, epithelial cells were grown to 80-90% confluency on coverslips in DMEM with 10% FBS and 1% penicillin/streptomycin. Three hours prior to infection, epithelial cells were incubated in DMEM with 0.1% glucose and without serum and antibiotics. To infect cells, mCherry-expressing bacteria were subcultured in DMEM with 0.1% glucose to mid-log phase in the presence of 2-AG or the vehicle control. Epithelial cells were then infected at an MOI of 10-30. At the indicated time points, the samples were washed, fixed, permeabilized and stained with FITC-labeled phalloidin and DAPI. Images for pedestal enumeration were taken using the Zeiss LSM780 confocal/multiphoton microscope at the UT Southwestern Live Cell Imaging Core Facility. Pedestal formation was quantified in two ways—as the number of pedestals per infected cell and the percentage of epithelial cells that contain pedestals.

Epithelial invasion assay. S. Typhimurium epithelial invasion assays were performed on HeLa cells as previously described.³⁰ Briefly, epithelial cells were grown to 80-90% confluency in DMEM with 10% FBS and 1% penicillin/streptomycin. Three hours prior to infection, epithelial cells were serum starved and incubated in the absence of antibiotics. To infect cells, S. Typhimurium was subcultured in LB to OD600=1.0 in the presence of 2-AG or the vehicle control. Epithelial cells were then infected at an MOI of 10 for 1 hour, followed by incubation with gentamicin for 1 hour to remove extracellular bacteria. Epithelial cells were then lysed with triton X-100 and intracellular bacteria were enumerated by quantitative bacterial culture.

Macrophage infection. S. Typhimurium infections of J774.1 murine macrophages were performed as previously described.³¹ Briefly, macrophages were infected with opsonized S. Typhimurium at an MOI of 100 for 30 minutes, followed by treatment with 40 μg/ml of gentamicin for 1 hour to kill extracellular bacteria. Macrophages were lysed with 1% Triton X-100 to release intracellular bacteria for enumeration by quantitative bacterial culture. Where indicated, 6 μM of 2-AG and/or the cannabinoid receptor 1 inhibitor AM251 (Tocris) were added to overnight cultures and at the start of infection.

CpxA and QseC purification. E. coli (B121 DE3) harboring pET21a-CpxA (His-tagged) or pVS155 (pBAD-QseC-MycHis) were grown until stationary phase (O.D ˜0.7-0.8). Cultures were induced with 0.1 mM IPTG or arabinose and grown further at 18° C. overnight. The bacterial cultures were spun down at 10000 rpm, 4° C. for 30 min and the pellets were stored at −80° C. until further use. The bacterial pellets were resuspended in Lysis buffer (50 mM Tris pH 8.0, 150 mM NaCl, 2% Lauryldimethylamine N-oxide (LDAO), 10% glycerol) and protease inhibitor cocktail (PIC) was added. The lysates were sonicated for 30 s ON and 30 s OFF for 8 min at 85% amplitude, followed by centrifugation at 10000 rpm, 4° C. for 30 min. The supernatants were filtered using 0.22 μm filter tube. 500 μl of Ni-NTA beads (Thermo Scientific—Product #88221) were added to 40 ml of lystate and kept on shaker for up to 2 hrs at 4° C. to allow protein binding to the Ni-beads. The bound lysates were passed through 5 ml disposable columns (Qiagen—Product #1018597) twice. The column was washed with 5 column volume (CV) wash buffer (50 mM Tris pH 8.0, 150 mM NaCl, 30 mM Imidazole). Proteins were eluted from the column by adding elution buffer (50 mM Tris pH8.0, 150 mM NaCl, 250 mM Imidazole). The eluted proteins were concentrated down using Amicon Ultra 15 Centrifugal Filter Units (10 kDa) and excess imidazole was removed by adding (50 mM Tris pH 8.0 and 150 mM NaCl) as a dialyzing buffer. Protein concentrations were estimated using Bradford assay and were immediately loaded on liposomes for autophosphorylation assays.

Autophosphorylation assays. These experiments were performed as previously described.³² Liposomes were reconstituted as described.³² Briefly, 50 mg of E. coli phospholipids (Avanti Polar Lipids, 20 mg/ml in chloroform) were evaporated and then dissolved into 5 ml phosphate buffer containing 80 mg N-octyl-β-D-glucopyranoside. The solution was dialyzed overnight against phosphate buffer. The resulting liposome suspension underwent freeze/thaw in liquid N2. The liposomes were stored at −80° C. until further use. The liposomes (1 ml) were then destabilized by the addition of 5.8 mg dodecylmaltoside, and CpxA-His was added in the ratio of 40:1, stirring at room temperature for 10 minutes. 58 mg of Biobeads were then added to remove the detergent, and the resulting solution was allowed to incubate at 4° C. overnight. The supernatant was then incubated with fresh Biobeads (58 mg) for another 1 hour. The resulting liposomes containing reconstituted CpxA-His was used for autophosphorylation experiments. 10 μl of the CpxA-His liposome was adjusted to 10 mM MgCl2 and 1 mM DTT, and 500 μM of indole, 10 μM 2-AG and 8 μM AA or no signal, freeze/thawed rapidly in liquid N2, and kept at room temperature for 1 hour. 0.25 μl of [γ³²P] ATP was added to each reaction. At each time point, 2 μl of 5×SDS loading buffer was added to stop the reaction. The samples were run on 12% SDS-PAGE according to standard procedures and visualized via phosphoimager (Typhoon FLA 9500, GE).

2-AG measurements. All solvents were either HPLC or LC/MS grade and purchased from Sigma-Aldrich (St Louis, Mo., USA). All lipid extractions were performed in 16×100 mm glass tubes with PTFE-lined caps (Fisher Scientific, Pittsburgh, Pa., USA). Glass Pasteur pipettes and solvent-resistant plasticware pipette tips (Mettler-Toledo, Columbus, Ohio, USA) were used to minimize leaching of polymers and plasticizers.

Approximately 100 mg of tissue was weighed and added to 1 mL of methanol/dichloromethane (1:2, v/v) in a 2.0-mL pre-filled Bead Ruptor tube (2.8 mm ceramic beads, Omni International, Kennesaw, Ga., USA). Tissue was homogenized with a Bead Ruptor (Omni International) for 50 s (5.5 mps, 3 cycles, 10 sec/cycle, 5 s dwell time). The homogenates were transferred to glass tubes and diluted to a final concentration of 20 mg/mL using methanol/dichloromethane (1:2, v/v).

Fatty Acid Profiling by GC-MS: Fatty acid profiles were generated by a modified GC-MS method previously described by Quehenberger et al.⁴¹ Briefly, 0.5 mg of liver homogenate was to a glass tube where 1 mL of MeOH with 50 mM HCl, 1 mL of water, 1 mL of iso-octane, and 100 uL of 0.5 μg/mL of FA(20:4 ω6{2H₈}) fatty acid standard in methanol were added. The mixture was vortexed for 5 minutes in a tube shaker and centrifuged at 2671×g for an additional 5 minutes. The organic phase was collected to a fresh glass tube. The procedure was repeated two times and the organic phases collected were pooled together and dried under N2. Dried lipid extract was resuspendend in 50 μL of 1% triethylamine in acetone, and derivatized with 50 μL of 1% pentafluorobenzyl bromide (PFBBr) in acetone at room temperature for 25 min in capped glass tubes. Solvents were dried under N2, and samples were resuspendend in 500 μL of isooctane. Samples were analyzed using an Agilent 7890/59750 (Santa Clara, Calif., USA) by electron capture negative ionization (ECNI) equipped with a DB-5MS column (40 m×0.180 mm with 0.18 μm film thickness) from Agilent. Hydrogen (carrier gas) flow rate was 1.6 mL/min and injection port temperature was set at 300° C. Sample injection volume was 1 μL. Initial oven temperature was set at 150° C., and then increased to 200° C. at a 25° C./min, followed by an increase of 8° C./min until a temperature of 300° C. was reached and held for 2.2 minutes, for a total run time was 16.7 min. Fatty acids were analyzed in selected ion monotoring (SIM) mode. The FA data was normalized to the internal standard. Data was processed using MassHunter software (Agilent).

LC-MS/MS analysis: Aliquots equivalent to 0.5 mg of homogenized tissue were transferred to fresh glass tubes for liquid-liquid extraction (LLE). The samples were dried under N2 and extracted by Bligh/Dyer;⁴² 1 mL each of chloroform, methanol, and water were added to a glass tube containing the sample. The mixture was vortexed and centrifuged at 2671×g for 5 min, resulting in two distinct liquid phases. The organic phase was collected to a fresh glass tube with a Pasteur pipette and dried under N2. Samples were resuspendend in Hexane.

The monoacylglycerrols (MAG) were analyzed by LC-MS/MS using a SCIEX QTRAP 6500+ equipped with a Shimadzu LC-30AD (Kyoto, Japan) HPLC system and a 150×2.1 mm, 5 μm Supelco Ascentis silica column (Bellefonte, Pa., USA). Samples were injected at a flow rate of 0.3 ml/min at 2.5% solvent B (methyl tert-butyl ether) and 97.5% Solvent A (Hexane). Solvent B is increased to 5% during 3 minutes and then to 60% over 6 minutes. Solvent B is decreased to 0% during 30 seconds while Solvent C (90:10 (v/v) Isopropanol-water) is set up at 20% and increased to 40% during the following 11 minutes. Solvent C is increased to 50% during 6 minutes. The system was held at 50% of solvent C during 5 minutes prior to re-equilibration at 2.5% of solvent B for 15 minutes. Solvent D (95:5 (v/v) Acetonitrile-water with 10 mM Ammonium acetate) was infused post-column at 0.03 ml/min. Column oven temperature was set to 25° C. Data was acquired in positive mode in positive ion mode using multiple reaction monitoring (MRM) of the m/z transitions 379.3→287.3, 331.3→239.2, 359.3→264.2, 357.3→265.2, 355.3→263.2, 387.2→295.2, 383.3→291.2, 381.3→289.2 and 403.4→311.2. The LC-MS data was analyzed using MultiQuant software (SCIEX). Electrospray ionization source parameters were, GS1 40, Cur 20, temperature 150° C., declustering potential 60, and collision energy 25. GS1 and 2 were zero-grade air while Cur and CAD gas was nitrogen.

Statistical analysis. P-values were calculated using Student's t test when 2 experimental groups were compared and one-way ANOVA with Bonferroni multiple comparison post test when 3 or more experimental groups were compared. All enumeration of bacteria by serial dilution and plating was log transformed to normalize the data. For microscopy analysis and animal studies, p-values were calculated using Mann-Whitney when 2 experimental groups were compared and Kruskal-Wallis test with the Dunn's post test when 3 or more experimental groups were compared.

Cannabidiol treatment of bacteria infected mice. C57BL/6J-WT adult-male mice were used to examine the in vivo effects of CBD-treatment on the virulence of type three secretion system pathogens. All mice except for the negative control were inoculated with ˜1×10⁹ CFUs of Citrobacter rodentium grown overnight in LG-DMEM without shaking. The mice were infected through sterile oral gavage conducted in an ABSL2 facility according to the rules established by Department of Comparative Medicine (DCM) at Oregon Health & Science University. The overnight cultures were concentrated to the proper infectious dose such that each mouse received ˜100 μL. Six mice received bacteria through a combined inoculate that contained solely the bacteria. These mice were infected using a separate sterile syringe and gavage to ensure no cross-contamination. Three of the mice from the combined inoculate were administered 100 μL or ˜3-4 mg/kg CBD 1-day post infection following fecal collection. Fecal collections occurred each day post-infection for seven days. All feces were weighed and solubilized in LB with 1% saponin and serial diluted for plate counts. Plating occurred in triplicate using technical replicates for each dilution. Plates contained 100 μg/ml nalidixic acid for selection. Colonies were assessed morphologically and by Gram stain to ensure counts were conducted on C. rodentium. The final mouse was administered CBD at the 1-day post infection point (without infection with the pathogen) and monitored as a negative control to ensure no C. rodentium appeared that would invalidate results due to potential cross-contamination or any potential artifact of the CBD solution. Treatment groups were all separated by cage and were sacrificed after the seven-day post-infection point.

Murine Pathogen Model. C57BL/6J-WT adult-male mice were used to examine the in vivo effects of CBD-treatment on the virulence of type three secretion system pathogens. All mice except for the negative control were inoculated with ˜1×10⁹ CFUs of Citrobacter rodentium grown overnight in LG-DMEM without shaking. The mice were infected through sterile oral gavage conducted in an ABSL2 facility according to the rules established by Department of Comparative Medicine (DCM) at Oregon Health & Science University. The overnight cultures were concentrated to the proper infectious dose such that each mouse received ˜100 μL. Six mice received bacteria through a combined inoculate that contained solely the bacteria. These mice were infected using a separate sterile syringe and gavage to ensure no cross-contamination. Three of the mice from the combined inoculate were administered 100 μL CBD 1-day post infection following fecal collection. Fecal collections occurred each day post-infection for seven days. All feces were weighed and solubilized in LB with 1% saponin and serial diluted for plate counts. Plating occurred in triplicate using technical replicates for each dilution. Plates contained 100 μg/ml nalidixic acid for selection. Colonies were assessed morphologically and by Gram stain to ensure counts were conducted on C. rodentium. The final mouse was administered CBD at the 1-day post infection point (without infection with the pathogen) and monitored as a negative control to ensure no C. rodentium appeared that would invalidate results due to potential cross-contamination or any potential artifact of the CBD solution. Treatment groups were all separated by cage and were sacrificed after the seven-day post-infection point.

### REFERENCES

1. Sartor, R. B. & Wu, G. D. Roles for Intestinal Bacteria, Viruses, and Fungi in Pathogenesis of Inflammatory Bowel Diseases and Therapeutic Approaches. Gastroenterology 152, 327-339.e4 (2017).
2. Bäumler, A. J. & Sperandio, V. Interactions between the microbiota and pathogenic bacteria in the gut. Nature 535, 85-93 (2016).
3. Turcotte, C., Chouinard, F., Lefebvre, J. S. & Flamand, N. Regulation of inflammation by cannabinoids, the endocannabinoids 2-arachidonoyl-glycerol and arachidonoyl-ethanolamide, and their metabolites. Journal of Leukocyte Biology 97, 1049-1070 (2015).
4. Cani, P. D. et al. Endocannabinoids—at the crossroads between the gut microbiota and host metabolism. Nat Rev Endocrinol 12, 133-143 (2016).
5. Côtes, K. et al. Characterization of an exported monoglyceride lipase from Mycobacterium tuberculosis possibly involved in the metabolism of host cell membrane lipids. Biochem. J. 408, 417-427 (2007).
6. Dhouib, R., Laval, F., Carrière, F., Daffé, M. & Canaan, S. A monoacylglycerol lipase from Mycobacterium smegmatis Involved in bacterial cell interaction. J Bacteriol 192, 4776-4785 (2010).
7. Alhouayek, M., Lambert, D. M., Delzenne, N. M., Cani, P. D. & Muccioli, G. G. Increasing endogenous 2-arachidonoylglycerol levels counteracts colitis and related systemic inflammation. The FASEB Journal 25, 2711-2721 (2011).
8. Schlosburg, J. E. et al. Chronic monoacylglycerol lipase blockade causes functional antagonism of the endocannabinoid system. Nat Neurosci 13, 1113-1119 (2010).
9. Kaper, J. B., Nataro, J. P. & Mobley, H. L. Pathogenic Escherichia coli. Nature Reviews Immunology 2, 123-140 (2004).
10. Deng, W. et al. Dissecting virulence: systematic and functional analyses of a pathogenicity island. Proc Natl Acad Sci USA 101, 3597-3602 (2004).
11. Di Sabatino, A. et al. The endogenous cannabinoid system in the gut of patients with inflammatory bowel disease. Mucosal Immunology 4, 574-583 (2011).
12. van der Stelt, M. et al. Oxygenated Metabolites of Anandamide and 2-Arachidonoylglycerol: Conformational Analysis and Interaction with Cannabinoid Receptors, Membrane Transporter, and Fatty Acid Amide Hydrolase. Journal of Medicinal Chemistry 45, 3709-3720 (2002).
13. Galán, J. E. & Curtiss, R. Cloning and molecular characterization of genes whose products allow Salmonella typhimurium to penetrate tissue culture cells. Proc Natl Acad Sci USA 86, 6383-6387 (1989).
14. Ochman, H., Soncini, F. C., Solomon, F. & Groisman, E. A. Identification of a pathogenicity island required for Salmonella survival in host cells. Proc Natl Acad Sci USA 93, 7800-7804 (1996).
15. Martins, M. Salmonella—host interactions—modulation of the host innate immune system. 1-11 (2014). doi:10.3389/fimmu.2014.00481/abstract
16. Clarke, M. B., Hughes, D. T., Zhu, C., Boedeker, E. C. & Sperandio, V. The QseC sensor kinase: a bacterial adrenergic receptor. Proc Natl Acad Sci USA 103, 10420-10425 (2006).
17. Rasko, D. A. et al. Targeting QseC Signaling and Virulence for Antibiotic Development. Science 321, 1078-1080 (2008).
18. Curtis, M. M. et al. QseC inhibitors as an antivirulence approach for Gram-negative pathogens. mBio 5, e02165 (2014).
19. Moreira, C. G. et al. Bacterial Adrenergic Sensors Regulate Virulence of Enteric Pathogens in the Gut. mBio 7, (2016).
20. Rooks, M. G. et al. QseC inhibition as an antivirulence approach for colitis-associated bacteria. Proceedings of the National Academy of Sciences 114, 142-147 (2017).
21. Szabo, B. & Schlicker, E. Effects of cannabinoids on neurotransmission. Handb Exp Pharmacol 327-365 (2005).
22. Sperandio, V., Torres, A. G. & Kaper, J. B. Quorum sensing Escherichia coli regulators B and C (QseBC): a novel two-component regulatory system involved in the regulation of flagella and motility by quorum sensing in E. coli. Mol Microbiol 43, 809-821 (2002).
23. Mechoulam, R. et al. Identification of an endogenous 2-monoglyceride, present in canine gut, that binds to cannabinoid receptors. Biochem Pharmacol 50, 83-90 (1995).
24. DiRusso, C. C. & Black, P. N. Bacterial long chain fatty acid transport: gateway to a fatty acid-responsive signaling system. J Biol Chem 279, 49563-49566 (2004).
25. K. A. Datsenko, B. L. Wanner, One-step inactivation of chromosomal genes in Escherichia coli K-12 using PCR products. Proc Natl Acad Sci USA 97, 6640-6645 (2000); published online EpubJun 6
26. J. C. Onyiah et al., Carbon monoxide and heme oxygenase-1 prevent intestinal inflammation in mice by promoting bacterial clearance. Gastroenterology. 144, 789-798 (2013).
27. D. L. Gibson et al., Toll-like receptor 2 plays a critical role in maintaining mucosal integrity during Citrobacter rodentium-induced colitis. Cell. Microbiol. 0, 071003010119001-??? (2007).
28. K. G. Jarvis et al., Enteropathogenic Escherichia coli contains a putative type III secretion system necessary for the export of proteins involved in attaching and effacing lesion formation. Proc Natl Acad Sci USA. 92, 7996-8000 (1995).
29. S. Knutton, M. M. McConnell, B. Rowe, A. S. McNeish, Adhesion and ultrastructural properties of human enterotoxigenic Escherichia coli producing colonization factor antigens III and IV. Infect Immun. 57, 3364-3371 (1989).
30. B. B. Finlay, S. Ruschkowski, S. Dedhar, Cytoskeletal rearrangements accompanying salmonella entry into epithelial cells. J. Cell. Sci. 99 (Pt 2), 283-296 (1991).
31. C. G. Moreira, D. Weinshenker, V. Sperandio, QseC mediates Salmonella enterica serovar typhimurium virulence in vitro and in vivo. Infect Immun. 78, 914-926 (2010).
32. M. B. Clarke, D. T. Hughes, C. Zhu, E. C. Boedeker, V. Sperandio, The QseC sensor kinase: a bacterial adrenergic receptor. Proc Natl Acad Sci USA. 103, 10420-10425 (2006).
33. V. Sperandio, A. G. Torres, J. B. Kaper, Quorum sensing Escherichia coli regulators B and C (QseBC): a novel two-component regulatory system involved in the regulation of flagella and motility by quorum sensing in E. coli. Mol Microbiol. 43, 809-821 (2002).
34. R. Pifer, R. M. Russell, A. Kumar, M. M. Curtis, V. Sperandio, Redox, amino acid, and fatty acid metabolism intersect with bacterial virulence in the gut. Proceedings of the National Academy of Sciences. 83, 201813451 (2018).
35. N. C. Reading, D. A. Rasko, A. G. Torres, V. Sperandio, The two-component system QseEF and the membrane protein QseG link adrenergic and stress sensing to bacterial pathogenesis. Proceedings of the National Academy of Sciences. 106, 5889-5894 (2009).
36. A. R. Pacheco et al., Fucose sensing regulates bacterial intestinal colonization. Nature. 492, 113-117 (2012).
37. S. K. Hoiseth, B. A. Stocker, Aromatic-dependent Salmonella typhimurium are non-virulent and effective as live vaccines. Nature. 291, 238-239 (1981).
38. J. E. Galán, R. Curtiss, Cloning and molecular characterization of genes whose products allow Salmonella typhimurium to penetrate tissue culture cells. Proc Natl Acad Sci USA. 86, 6383-6387 (1989).
39. S. W. Barthold, G. L. Coleman, P. N. Bhatt, G. W. Osbaldiston, A. M. Jonas, The etiology of transmissible murine colonic hyperplasia. Lab. Anim. Sci. 26, 889-894 (1976).
40. M. M. Curtis et al., The Gut Commensal Bacteroides thetaiotaomicron Exacerbates Enteric Infection through Modification of the Metabolic Landscape. Cell Host Microbe. 16, 759-769 (2014).
41. O. Quehenberger, A. M. Armando, E. A. Dennis. High sensitivity quantitative lipidomics analysis of fatty acids in biological samples by gas chromatography-mass spectrometry. Biochim Biophys Acta. 1811, 648-656 (2011).
42. E. G. Blight, W. J. Dyer. A rapid method of total lipid extraction and purification. Canadian Journal of Biochemistry and Physiology. 37, 911-917 (1959).
43. L. Spiga, M. G. Winter et al., An oxidative central metabolism enables Salmonella to utilize microbiota-derived succinate. Cell Host and Microbe. 22, 291-301 (2017).
44. K. Doheny, “Marijuana, Hemp, CBD: What's Legal and Where,” WebMD. [Online]. Available: https://www.webmd.com/pain-management/news/20190108/marijuana-hemp-cbd-whats-legal-and-where. [Accessed: 28 Jan. 2019].
45. O. of the Commissioner, “Press Announcements—FDA approves first drug comprised of an active ingredient derived from marijuana to treat rare, severe forms of epilepsy.” [Online]. Available: https://www.fda.gov/NewsEvents/Newsroom/PressAnnouncements/ucm611046.htm. [Accessed: 28 Jan. 2019].
46. “WHO|Cannabidiol (compound of cannabis),” WHO. [Online]. Available: http://www.who.int/features/qa/cannabidiol/en/. [Accessed: 28 Jan. 2019].
47. R. G. Pertwee, “The diverse CB1 and CB2 receptor pharmacology of three plant cannabinoids: Δ9-tetrahydrocannabinol, cannabidiol and Δ9-tetrahydrocannabivarin,” Br. J. Pharmacol., vol. 153, no. 2, pp. 199-215, January 2008.
48. J. M. McPartland, M. Duncan, V. Di Marzo, and R. G. Pertwee, “Are cannabidiol and Δ(9)-tetrahydrocannabivarin negative modulators of the endocannabinoid system? A systematic review,” Br. J. Pharmacol., vol. 172, no. 3, pp. 737-753, February 2015.
49. M. M. Curtis et al., “QseC Inhibitors as an Antivirulence Approach for Gram-Negative Pathogens,” mBio, vol. 5, no. 6, pp. e02165-14, December 2014.
50. M. M. Kendall and V. Sperandio, “What a Dinner Party! Mechanisms and Functions of Interkingdom Signaling in Host-Pathogen Associations,” mBio, vol. 7, no. 2, pp. e01748-15, March/April 2016.

All references cited in this disclosure are incorporated by reference in their entirety.

It will be apparent to those having skill in the art that many changes may be made to the details of the above-described embodiments without departing from the underlying principles of the invention. The scope of the present invention should, therefore, be determined only by the following claims.

1-25. (canceled)

26. A method of reducing bacterial virulence in a subject with a gram negative bacterial infection, comprising:
administering to the subject a therapeutically effective amount of a pharmaceutical composition comprising a cannabinoid and a pharmaceutically acceptable carrier.

27. The method of claim 26, wherein the cannabinoid is selected from 2-arachidonoyl-glycerol or cannabidiol.

28. The method of claim 26, wherein the therapeutically effective amount of the cannabinoid is between about 1-30 mg/kg, about 1-25 mg/kg, about 1-20 mg/kg, about 1-15 mg/kg, about 1-10 mg/kg, about 1-5 mg/kg, or about 3-4 mg/kg body weight.

29. The method of claim 26, wherein the gram negative bacterial infection is a gastrointestinal tract infection.

30. (canceled)

31. The method of claim 26, wherein the gram negative bacterial infection is selected from at least one of a Salmonella Typhimurium infection or an enterohemorrhagic Escherichia coli infection.

32. The method of claim 26, wherein the pharmaceutical composition is administered in a plurality of doses.

33. (canceled)

34. The method of claim 26, wherein the subject is a human.

35. The method of claim 26, wherein the subject is a domesticated animal.

36-38. (canceled)

39. The method of claim 31, further comprising determining bacterial virulence, wherein bacterial virulence is determined by evaluating at least one of the activity of the bacterial type 3 secretion system, the expression of a bacterial pathogenicity island, the secretion of bacterial effector proteins, lesion formation, or colonic tissue damage.

40. (canceled)

41. The method of claim 26, further comprising the step of administering to the subject an antibiotic.

42. The method of claim 26, wherein the pharmaceutical composition further comprises one or more antibiotics.

43. The method of claim 26, wherein the pharmaceutical composition is administered orally.

44-46. (canceled)

47. A method for reducing bacterial virulence in a subject having an antibiotic-resistant gram negative bacterial infection, comprising:
administering to the subject a therapeutically effective amount of a pharmaceutical composition comprising a cannabinoid and a pharmaceutically acceptable carrier to reduce a pathological effect or symptom of the gram negative bacterial infection.

48. The method of claim 47, wherein the bacterial infection is multidrug resistant.

49. The method of claim 47, wherein the bacterial infection is a Carbapenem-resistant Enterobacteriaceae (CRE) infection, or an extended-spectrum Beta-lactamase producing Enterobacteriaceae infection.

50-53. (canceled)

54. The method of claim 47, wherein the cannabinoid is selected from 2-arachidonoyl-glycerol or cannabidiol.

55. (canceled)

56. The method of claim 47, wherein the gram negative bacterial infection is a gastrointestinal tract infection.

57. The method of claim 47, wherein the pharmaceutical composition is administered in a plurality of doses.

58. (canceled)

59. The method of claim 47, wherein the subject is a human.

60. The method of claim 47, wherein the subject is a domesticated animal.

61-62. (canceled)
